(19) United States
(12) Patent Application Publication
(10) Pub. No.: US 2011/0034400 A1
(43) Pub. Date: Feb. 10, 2011

(54) COMPOSITIONS AND METHODS FOR ANALYZING BACTERIAL ADHERENCE AND ANTI-ADHERENCE TO MUCUS, EPITHELIAL CELLS AND OTHER CELLS

(75) Inventors: Karl A. Dawson, Lexington, KY (US); Colm Moran, La Ciotat (FR); Juha Apajalahti, Helsinki (FI); Marko Lauraeus, Nummela (FI)

Correspondence Address: Casimir Jones, S. C., 2275 Deming Way, Suite 310, Middleton, WI, 53562, US

(73) Assignee: ALLTECH, INC., Nicholasville, KY (US)

(21) Appl. No.: 12/832,746

(22) Filed: Jul. 8, 2010

Related U.S. Application Data
(60) Provisional application No. 61/223,755, filed on Jul. 8, 2009.

(51) Int. Cl. A61K 38/14; G01N 33/53; C07K 14/00; A61P 43/00

(52) U.S. Cl. 514/20.9; 435/7.92; 530/395

(57) ABSTRACT

The present invention generally relates to methods for detecting, identifying, and measuring bacterial adherence to mucus and epithelial cells. In particular, the present invention provides assays for detecting and identifying the presence or absence of bacterial adherence to mucus (epithelial cells (e.g., present in the intestines), or other portion of an animal where bacteria may be present, and methods for identifying and characterizing (e.g., the efficacy of) modulators of bacterial adherence to mucus and epithelial cells, or other portion of the animal where bacteria may be present.

### CROSS REFERENCE TO RELATED APPLICATIONS

The present application claims priority to U.S. Provisional Application Ser. No. 61/223,755 filed Jul. 8, 2009, the entirety of which is herein incorporated by reference.

### FIELD OF THE INVENTION

The present invention generally relates to methods for detecting, identifying, and measuring bacterial adherence to mucus and epithelial cells. In particular, the present invention provides assays for detecting and identifying the presence or absence of bacterial adherence to mucus (epithelial cells (e.g., present in the intestines), or other portion of an animal where bacteria may be present, and methods for identifying and characterizing (e.g., the efficacy of) modulators of bacterial adherence to mucus and epithelial cells, or other portion of the animal where bacteria may be present.

### BACKGROUND OF THE INVENTION

The epithelial cells in the small intestine, the respiratory tract, the urinary tract, and the reproductive tracts of animals are covered by a relatively thick layer of mucus which comprises mucin, many small associated proteins, glycoproteins, lipids, and glycolipids. The epithelial cells and mucus contain receptors that recognize specific bacterial adhesion proteins. Adhesion or close association of bacteria to the epithelial cells may contribute to colonization as well as to bacterial pathogenicity. In addition, bacterial adhesion to intestinal mucus and epithelia appears important for individual stability within the microbial flora.

### SUMMARY OF THE INVENTION

The present invention generally relates to methods for detecting, identifying, and measuring bacterial adherence and anti-adherence to mucus and cells (e.g., epithelial cells). In particular, the present invention provides assays for detecting and identifying bacterial adherence to mucus (e.g., intestinal mucosal lining) and epithelial cells, and methods for identifying modulators of bacterial adherence to mucus and epithelial cells. The assays are non-radioactive, microbiologically safe, as well as stable, easily transported, and easily stored.

Accordingly, in some embodiments, the present invention provides kits comprising a non-radioactive enzyme-linked immunosorbent assay (ELISA) for the assay of bacterial adherence with mucus and/or epithelial cells. The kits are not limited to particular components. In some embodiments, the kits comprise a solid support having mucus or epithelial cells coated thereon, a sample comprising bacteria, a primary antibody specific for the bacteria, and a detectably labeled secondary antibody specific for the primary antibody bound to the bacteria. In some embodiments, the kits comprise a substrate which allows the visualization of the detectably labeled secondary antibody. In some embodiments, the detectably labeled secondary antibody comprises an enzyme label. In some embodiments, the substrate is a composition for providing a colorimetric, fluorimetric or chemiluminescent signal in the presence of the enzyme label. In some embodiments, the detectably labeled secondary antibody comprises pig anti-IgG immunoglobulins coupled to peroxidase. In some embodiments, the colorimetric composition is 3,3′,5,5′-tetramethylbenzidine. In some embodiments, the solid support is a 96-well plate. In some embodiments, the bacteria is E. coli bacteria. Examples of mucus include, but are not limited to, pig proximal ileum mucus, pig distal colon mucus, broiler duodenum mucus and broiler caecum mucus. Examples of primary antibodies include but are not limited to HRP-conjugated polyclonal antibodies specific to E. coli O and K antigenic serotypes, polyclonal antibodies specific to E. coli O and K antigenic serotypes. Examples of secondary antibodies include but are not limited to affinity purified Rabbit anti-Goat IgG-HRP, affinity purified Rabbit anti-Goat IgG-AP, polyclonal FITC-conjugated antibodies to Goat IgG (H&L), Streptavidin-Alkaline Phosphatase from Streptomyces avidinii, and Streptavidin-Peroxidase from Streptomyces avidinii.

In certain embodiments, the present invention provides methods for measuring adherence and anti-adherence between bacteria and mucus and bacteria and epithelial cells. The methods are not limited to particular techniques for measuring adherence between bacteria and mucus and bacteria and epithelial cells. In some embodiments, the methods comprise providing a sample comprising bacteria and mucus, and combining the sample comprising bacteria and mucus within a non-radioactive colorimetric assay under conditions such that adherence between the bacteria and the mucus is measured. In some embodiments, the non-radioactive colorimetric assay is an ELISA assay. In some embodiments, the conditions comprise adding primary antibodies specific for the bacteria bound with the mucus, epithelial or other cells, and adding detectably labeled secondary antibodies specific for the primary antibodies bound with the bacteria. In some embodiments, the methods comprise adding a substrate which allows the visualization of the detectably labeled secondary antibodies bound with the primary antibodies. In some embodiments, mucus is coated onto a microtitre plate. In some embodiments, the detectably labeled secondary antibody comprises an enzyme label. In some embodiments, the substrate is a composition for providing a colorimetric, fluorimetric or chemiluminescent signal in the presence of the enzyme label. In some embodiments, the detectably labeled secondary antibody comprises pig anti-IgG immunoglobulins coupled to peroxidase. In some embodiments, the colorimetric composition is 3,3′,5,5′-tetramethylbenzidine. In some embodiments, the bacteria is E. coli bacteria. The methods are not limited to particular primary or secondary antibodies. In some embodiments, examples of primary antibodies include but are not limited to an HRP-conjugated polyclonal antibody specific to E. coli O and K antigenic serotypes, and a polyclonal antibody specific to E. coli O and K antigenic serotypes. Examples of secondary antibodies include but are not limited to an affinity purified Rabbit anti-Goat IgG-HRP, an affinity purified Rabbit anti-Goat IgG-AP, a polyclonal FITC-conjugated antibody to Goat IgG (H&L), Streptavidin-Alkaline Phosphatase from Streptomyces avidinii, and Streptavidin-Peroxidase from Streptomyces avidinii.

In certain embodiments, the present invention provides methods for identifying an agent that modulates adherence between bacteria and mucus, comprising providing a sample comprising bacteria, mucus, and an agent, and combining the sample comprising bacteria, the mucus, and the agent within a non-radioactive colorimetric assay under conditions such that adherence between the bacteria and the mucus is measured. The methods further comprise comparing the bacterial adherence in the presence and absence of the agent, and identifying the agent as a modulator of adherence between the bacteria and the mucus if the measured adherence is higher or lower than adherence between the bacteria and the mucus in the absence of the agent. In some embodiments, the non-radioactive colorimetric assay is an ELISA assay. In some embodiments, the conditions comprise adding primary antibodies specific for the bacteria bound with the mucus. In some embodiments, the conditions comprise adding detectably labeled secondary antibodies specific for the primary antibodies bound with the bacteria. In some embodiments, the conditions comprise adding a substrate which allows the visualization of the detectably labeled secondary antibodies bound with the primary antibodies. In some embodiments, the mucus are coated onto a microtitre plate. In some embodiments, the detectably labeled secondary antibody comprises an enzyme label. In some embodiments, the substrate is a composition for providing a colorimetric, fluorimetric or chemiluminescent signal in the presence of the enzyme label. In some embodiments, the detectably labeled secondary antibody comprises pig anti-IgG immunoglobulins coupled to peroxidase. In some embodiments, the colorimetric composition is 3,3′,5,5′-tetramethylbenzidine. In some embodiments, the bacteria are E. coli bacteria. Examples of primary antibodies include, but are not limited to, an HRP-conjugated polyclonal antibody specific to E. coli O and K antigenic serotypes, a polyclonal antibody specific to E. coli O and K antigenic serotypes. Examples of secondary antibodies include, but are not limited to, an affinity purified Rabbit anti-Goat IgG-HRP, an affinity purified Rabbit anti-Goat IgG-AP, a polyclonal FITC-conjugated antibody to Goat IgG (H&L), Streptavidin-Alkaline Phosphatase from Streptomyces avidinii, and Streptavidin-Peroxidase from Streptomyces avidinii. In some embodiments, the agent is selected from a list consisting of a naturally occuring molecule, a synthetically derived molecule, and a recombinantly derived molecule.

In certain embodiments, the present invention provides compositions comprising an agent, wherein the agent is a modulator of bacterial adherence with mucus, and wherein the agent is identified through a process comprising providing i) a sample comprising bacteria, ii) mucus, iii) an agent; combining the sample comprising bacteria, the mucus, and the agent within a non-radioactive colorimetric assay under conditions such that adherence between the bacteria and the mucus is measured; comparing the bacterial adherence in the presence and absence of the agent; and identifying the agent as a modulator of adherence between the bacteria and the mucus if the measured adherence is higher or lower than adherence between the bacteria and the mucus in the absence of the agent. In some embodiments, the composition is within a foodstuff configured for consumption by a subject selected from the group consisting of livestock, companion animals, fish, and shellfish.

### DESCRIPTION OF THE DRAWINGS

FIG. 1 shows the effect of mucus concentration (mg protein/ml) of E. coli ALI 84 and AL1446 adherence, as measured by radioactively-labeled bacteria.

FIG. 2 shows the effect of primary antibodies on the adherence of E. coli AL184, as measured by scintillation counter with radioactively-labeled bacteria. Primary antibodies: HRP=HRP-conjugated anti-E. coli; BP2022=unconjugated anti-E. coli, Biotin=biotin-conjugated anti-E. coli.

FIG. 3 shows the effect of primary antibodies on the adherence of E. coli AL1446, as measured by scintillation counter with radioactive-labeled bacteria. Primary antibodies: HRP=HRP-conjugated anti-E. coli; BP2022=unconjugated anti-E. coli, Biotin=biotin-conjugated anti-E. coli.

FIG. 4 shows color development of three different 3,3′,5,5′-tetramethylbenzidine (TMB) ELISA substrates when incubated with E. coli-bacteria (strains AL184 and AL1446).

FIG. 5 shows color development of p-nitrophenyl phosphate (pNPP) and 2,2′-Azino-bis(3ethylbenzothiazoline-6-sulfonic acid) (AzBTS) ELISA substrates when incubated with E. coli strains AL184 and AL1446. E=ABTS microwell enhancer.

FIG. 6 shows color development of different ELISA substrates when incubated in mucus coated wells. The photo was taken at 60 min, a weak signal was observed in the six positive (yellow) wells at 15 min.

FIG. 7 shows the plate layout when testing unspecific binding of antibodies to mucus or plate. BP2022=anti-E. coli-primary antibody; BP2022HRP=peroxidase conjugated anti-E. coli-primary antibody; Biotin=biotin conjugated anti-E. coli-primary antibody. HRP=peroxidase conjugated secondary antibody; StrHRP=peroxidase conjugated streptavidin. AP=alkaline phosphatase conjugated secondary antibody; StrAP=streptavidin conjugated secondary antibody antibody. No bacteria were used in this experiment.

FIG. 8 shows binding of antibodies to mucus or plate. Plate layout is described in FIG. 7.

FIG. 9 shows non-specific binding test with primary and secondary antibodies. Primary antibodies: HRP=HRP-conjugated 1st ab, BP2022=non-conjugated polyclonal anti-E. coli 1st antibody; Biotin=biotin-conjugated anti-E. coli 1st antibody. Secondary antibodies: HRP=HRP-conjugated IgG; StrHRP=HRP-labeled streptavidin. No bacteria were used in this experiment.

FIG. 10 shows unspecific binding test with primary and secondary antibodies.

Plate layout is described in FIG. 9. Primary antibodies: HRP=HRP-conjugated 1sl ab, BP2022=non-conjugated polyclonal anti-E. coli; Biotin=biotin-conjugated anti-E. coli. Secondary antibodies: HRP=HRP-conjugated IgG; Str.HRP=HRP-Iabeled streptavidin.

FIG. 11 shows a table describing conditions utilized for optimizing the dilution of antibodies and the number of bacteria/well. Primary antibody: HRP-conjugated primary antibody, no secondary antibody.

FIG. 12 shows data obtained from testing the dilution of antibodies and optimal number of bacteria/well. Primary antibody: HRP-conjugated primary antibody, no secondary antibody. Plate layout is shown in FIG. 11.

FIG. 13 shows a table describing conditions utilized for optimizing the dilution of antibodies and the number of bacteria/well. Primary antibody: biotin-conjugated anti-E. coli, secondary antibody: HRP-conjugated streptavidin.

FIG. 14 shows data obtained from testing the dilution of antibodies and optimal number of bacteria/well. Primary antibody: biotin-conjugated anti-E. coli, secondary antibody: HRP-conjugated streptavidin.

FIG. 15 shows ELISA absorbances with 10⁶-10⁸ bacteria added in the wells. A logarithmic trend line has been added.

FIG. 16 shows ELISA absorbances with 0-10⁶ of bacteria added in the wells.

FIG. 17 shows A) the effect of different concentrations of Bio-Mos on bacterial adherence. The effect is shown as percentage of absorbance when no Bio-Mos was added; and B) the effect of Bio-Mos on the adherence of E. coli strain AL184, measured with radioactively-labeled bacteria in scintillation counter.

FIG. 18 shows data from experiments testing the number of bacteria/well to find an optimal level for detecting adherence/attachment altering effects (e.g., using Bio-Mos).

FIG. 19 shows data from experiments testing the number of bacteria/well to find an optimal level for detecting adherence/attachment altering effects (e.g., using Bio-Mos).

FIG. 20 shows data related to primary antibody dilution for detecting differences between different concentrations of Bio-Mos.

FIG. 21 shows the effect of Bio-Mos in ELISA using different types of mucus and multiple Bio-Mos concentrations.

FIG. 22 shows Comparison of Bio-Mos effect in the radioactive attachment assay and ELISA procedure. Standard errors of the mean between replicate samples are shown as error bars.

FIG. 23 shows adherence of differently inactivated bacteria to mucus coated plates. Adherence was measured with and without Bio-Mos. Data for UV and DMSO-inactivated bacteria is not shown.

FIG. 24 shows adherence of bacteria to mucus on freshly coated plates according to the ELISA method.

FIG. 25 shows the effect of ethanol concentration in E. coli preservation liquid on the adherence of the bacteria on mucus. FIG. 26 shows adherence of bacteria with and without Bio-Mos on differently stored mucus coated plates.

FIG. 27 shows the Bio-Mos effect on adherence of ethanol inactivated E. coli on mucus coated, air-dried plates. Standard errors of the mean between replicates are shown as error bars.

FIG. 28 shows absolute plate-to-plate variation for different Bio-Mos test levels. Replicate assays (wells) of the same sample are shown as groups of bars.

FIG. 29 shows absolute plate-to-plate variation for different Bio-Mos test levels. The four panels of the figure represent assays carried out on four different days, but with a single batch of E. coli. Replicate assays (wells) of the same sample are shown as groups of bars.

FIG. 30 shows absolute plate-to-plate variation for different Bio-Mos test levels in different panels. The four sets of columns in each panel represent assays carried out on four different days, but with a single batch of E. coli.

FIG. 31 shows relative plate-to-plate variation for different Bio-Mos test levels. Columns represent means of the replicate test wells and the bars indicate standard errors of the mean. Assays were carried out on four different days, but with a single batch of E. coli. The two panels show the same data, but displayed differently to emphasize either plate-to-plate variation (upper panel) or the effect of Bio-Mos (lower panel).

FIG. 32 shows relative batch-to-batch variation for the effect of Bio-Mos. Columns of the upper panel represent means of the replicate test wells and the bars indicate standard errors of the mean. Assays were carried out totally independently starting from the medium and buffer preparations, and the cultivation of E. coli. The upper panel shows the measured signals and the lower panel the values relative to the control wells.

FIG. 33 shows number of replicate wells needed to detect indicated differences between the test treatments with the developed assay.

FIG. 34 shows signals measured for five (5) independent batches of bacterial preparation in the presence and absence of Bio-Mos (2 ng/ml).

FIG. 35 shows signals measured for five (5) independent batches of mucus plates in the presence and absence of Bio-Mos (2 ng/ml).

FIG. 36 shows signals of test after 1 and 2 weeks of storage.

FIG. 37 shows a vacuum sealed plate and ampoule in one embodiment of the invention.

### DEFINITIONS

To facilitate an understanding of the invention, a number of terms are defined below. As used herein, the term “mucus” refers to a relatively thick secretion produced by and covering portions of the digestive tract (e.g., produced by and covering the epithelial cells of the intestine). Mucus may comprise one or more components such as mucin, proteins, glycoproteins, lipids, and glycolipids. Mucus may also comprise one or more types of receptors (e.g., that recognize specific adhesion proteins). Adhesion and/or close association of bacteria to mucus and/or epithelial cells (e.g., via the mucus layer) may contribute to bacterial adhesion to intestinal mucus and/or epithelia (e.g., thereby playing a role in populations of bacteria that inhabit the gut). The present invention is not limited to any particular type of mucus or to mucus obtained from any particular source (e.g., type of animal) or location (e.g., part of the digestive tract (e.g., ileum (e.g., proximal, distal, etc.), duodenum, caecum, colon or other part of the digestive tract)).

As used herein, the terms “peptide,” “polypeptide” and “protein” all refer to a primary sequence of amino acids that are joined by covalent “peptide linkages.” In general, a peptide consists of a few amino acids, typically from 2-50 amino acids, and is shorter than a protein. The term “polypeptide” encompasses peptides and proteins. In some embodiments, the peptide, polypeptide or protein is synthetic, while in other embodiments, the peptide, polypeptide or protein are recombinant or naturally occurring. A synthetic peptide is a peptide that is produced by artificial means in vitro (i.e., was not produced in vivo).

The terms “sample” and “specimen” are used in their broadest sense and encompass samples or specimens obtained from any source. As used herein, the term “sample” is used to refer to biological samples obtained from animals (including humans), and encompasses fluids, solids, tissues, and gases. In some embodiments of this invention, biological samples include cerebrospinal fluid (CSF), serous fluid, urine, saliva, blood, and blood products such as plasma, serum and the like. However, these examples are not to be construed as limiting the types of samples that find use with the present invention.

As used herein, the terms “host” and “subject” refer to any animal, including but not limited to, human and non-human animals (e.g., dogs, cats, cows, horses, sheep, poultry, fish, crustaceans, etc.) that is studied, analyzed, tested, diagnosed or treated. As used herein, the terms “host,” “subject” and “patient” are used interchangeably, unless indicated otherwise.

As used herein, the term “antibody” (or “antibodies”) refers to any immunoglobulin that binds specifically to an antigenic determinant, and specifically binds to proteins identical or structurally related to the antigenic determinant that stimulated their production.

Thus, antibodies can be useful in assays to detect the antigen that stimulated their production. Monoclonal antibodies are derived from a single clone of B lymphocytes (i.e., B cells), and are generally homogeneous in structure and antigen specificity. Polyclonal antibodies originate from many different clones of antibody-producing cells, and thus are heterogenous in their structure and epitope specificity, but all recognize the same antigen. In some embodiments, monoclonal and polyclonal antibodies are used as crude preparations, while in preferred embodiments, these antibodies are purified. For example, in some embodiments, polyclonal antibodies contained in crude antiserum are used. Also, it is intended that the term “antibody” encompass any immunoglobulin (e.g., IgG, IgM, IgA, IgE, IgD, etc.) obtained from any source (e.g., humans, rodents, non-human primates, lagomorphs, caprines, bovines, equines, ovines, etc.).

As used herein, the term “antigen” is used in reference to any substance that is capable of being recognized by an antibody. It is intended that this term encompass any antigen and “immunogen” (i.e., a substance that induces the formation of antibodies). Thus, in an immunogenic reaction, antibodies are produced in response to the presence of an antigen or portion of an antigen. The terms “antigen” and “immunogen” are used to refer to an individual macromolecule or to a homogeneous or heterogeneous population of antigenic macromolecules. It is intended that the terms antigen and immunogen encompass protein molecules or portions of protein molecules, that contains one or more epitopes. In many cases, antigens are also immunogens, thus the term “antigen” is often used interchangeably with the term “immunogen.” In some preferred embodiments, immunogenic substances are used as antigens in assays to detect the presence of appropriate antibodies in the serum of an immunized animal.

As used herein, the terms “antigen fragment” and “portion of an antigen” and the like are used in reference to a portion of an antigen. Antigen fragments or portions typically range in size, from a small percentage of the entire antigen to a large percentage, but not 100%, of the antigen. However, in situations where “at least a portion” of an antigen is specified, it is contemplated that the entire antigen may also be present (e.g., it is not intended that the sample tested contain only a portion of an antigen). In some embodiments, antigen fragments and/or portions thereof, comprise an “epitope” recognized by an antibody, while in other embodiments these fragments and/or portions do not comprise an epitope recognized by an antibody. In addition, in some embodiments, antigen fragments and/or portions are not immunogenic, while in preferred embodiments, the antigen fragments and/or portions are immunogenic.

The terms “antigenic determinant” and “epitope” as used herein refer to that portion of an antigen that makes contact with a particular antibody variable region. When a protein or fragment (or portion) of a protein is used to immunize a host animal, numerous regions of the protein are likely to induce the production of antibodies that bind specifically to a given region or three-dimensional structure on the protein (these regions and/or structures are referred to as “antigenic determinants”). In some settings, antigenic determinants compete with the intact antigen (i.e., the “immunogen” used to elicit the immune response) for binding to an antibody.

The terms “specific binding” and “specifically binding” when used in reference to the interaction between an antibody and an antigen describe an interaction that is dependent upon the presence of a particular structure (i.e., the antigenic determinant or epitope) on the antigen. In other words, the antibody recognizes and binds to a protein structure unique to the antigen, rather than binding to all proteins in general (i.e., non-specific binding).

As used herein, the term “immunoassay” refers to any assay that uses at least one specific antibody for the detection or quantitation of an antigen. Immunoassays include, but are not limited to, Western blots, ELISAs, radio-immunoassays, and immunofluorescence assays.

As used herein, the term “ELISA” refers to enzyme-linked immunosorbent assay (or EIA). Numerous ELISA methods and applications are known in the art, and are described in many references (See, e.g., Crowther, “Enzyme-Linked Immunosorbent Assay (ELISA),” in Molecular Biomethods Handbook, Rapley et al. (eds.), pp. 595-617, Humana Press, Inc., Totowa, N.J. (1998); Harlow and Lane (eds.), Antibodies: A Laboratory Manual, Cold Spring Harbor Laboratory Press (1988); Ausubel et al. (eds.), Current Protocols in Molecular Biology, Ch. 11, John Wiley & Sons, Inc., New York (1994)). In addition, there are numerous commercially available ELISA test systems.

As used herein, the terms “reporter reagent,” “reporter molecule,” “detection substrate” and “detection reagent” are used in reference to reagents that permit the detection and/or quantitation of an antibody bound to an antigen. For example, in some embodiments, the reporter reagent is a colorimetric substrate for an enzyme that has been conjugated to an antibody. Addition of a suitable substrate to the antibody-enzyme conjugate results in the production of a colorimetric or fluorimetric signal (e.g., following the binding of the conjugated antibody to the antigen of interest). Other reporter reagents include, but are not limited to, radioactive compounds. This definition also encompasses the use of biotin and avidin-based compounds (e.g., including but not limited to neutravidin and streptavidin) as part of the detection system.

As used herein, the term “signal” is used generally in reference to any detectable process that indicates that a reaction has occurred, for example, binding of antibody to antigen. It is contemplated that signals in the form of radioactivity, fluorimetric or colorimetric products/reagents will all find use with the present invention. In various embodiments of the present invention, the signal is assessed qualitatively, while in alternative embodiments, the signal is assessed quantitatively.

As used herein, the term “solid support” is used in reference to any solid or stationary material to which reagents such as antibodies, antigens, and other test components are attached. For example, in an ELISA method, the wells of microtiter plates provide solid supports. Other examples of solid supports include microscope slides, coverslips, beads, particles, cell culture flasks, as well as many other suitable items.

As used herein, the term “effective amount” refers to the amount of a composition sufficient to effect beneficial or desired results. An effective amount can be administered and/or combined with another material in one or more administrations, applications or dosages and is not intended to be limited to a particular formulation or administration route.

As used herein, the terms “administration” and “administering” refer to the act of giving a drug, prodrug, or other agent, or therapeutic treatment (e.g., an agent identified as a modulator of bacterial adherence to mucus through use of the methods of the present invention) to a subject (e.g., a subject or in vivo, in vitro, or ex vivo cells, tissues, and organs). Exemplary routes of administration can be through the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., an agent identified as a modulator of bacterial adherence to mucus through use of the methods of the present invention and one or more other agents (e.g., a therapy known to treat pathogenic bacteria disorders) to a subject and/or material (e.g., a foodstuff (e.g., animal feed))). In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered and/or formulated at lower dosages than appropriate for their administration and/or formulation alone. Thus, co-administration is especially desirable in embodiments where the co-administration/co-formulation of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein “post-colonization treatment” or “post-application” refers to treatment after the removal of infectious disease.

As used herein “pre-application” and/or “prophylactic treatment” refers to treatments used as a preventative measure (e.g., to prevent infection and/or disease).

As used herein, the terms “disease” and “pathological condition” are used interchangeably to describe a state, signs, and/or symptoms that are associated with any impairment of the normal state of a living animal or of any of its organs or tissues that interrupts or modifies the performance of normal functions, and may be a response to environmental factors (such as malnutrition, industrial hazards, or climate), to specific infective agents (such as worms, bacteria, or viruses), to inherent defect of the organism (such as various genetic anomalies, or to combinations of these and other factors).

As used herein, the term “suffering from disease” refers to a subject (e.g., an animal or human subject) that is experiencing a particular disease. It is not intended that the present invention be limited to any particular signs or symptoms, nor disease. Thus, it is intended that the present invention encompass subjects that are experiencing any range of disease (e.g., from sub-clinical manifestation to full-blown disease) wherein the subject exhibits at least some of the indicia (e.g., signs and symptoms) associated with the particular disease.

As used herein, the term “toxic” refers to any detrimental or harmful effects on a subject, a cell, or a tissue as compared to the same cell or tissue prior to the administration of the toxicant.

As used herein, the term “functional feed ingredient” or “functional feed additive” refers to the combination of an active agent (e.g., an agent identified as a modulator of bacterial adherence with mucus) with a carrier, inert or active, making the composition especially suitable for diagnostic or therapeutic use in vitro, in vivo or ex vivo. .

As used herein, the term “carrier” refers to any standard carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), corn cob, dried distillers grains, wheat bran, yeast (e.g., whole spent yeast), yeast components (e.g., yeast cell wall extract), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference).

As used herein, the term “digest” refers to the conversion of food, feedstuffs, or other organic compounds into absorbable form; to soften, decompose, or break down by heat and moisture or chemical action.

As used herein, “digestive system” refers to a system (including gastrointestinal system) in which digestion can or does occur.

As used herein, the term “feedstuffs” refers to material(s) that are consumed by animals and contribute energy and/or nutrients to an animal's diet. Examples of feedstuffs include, but are not limited to, Total Mixed Ration (TMR), forage(s), pellet(s), concentrate(s), premix(es) coproduct(s), grain(s), distiller grain(s), molasses, fiber(s), fodder(s), grass(es), hay, kernel(s), leaves, meal, soluble(s), and supplement(s).

As used herein, the term “animal” refers to those of kingdom Animalia. This includes, but is not limited to livestock, farm animals, domestic animals, pet animals, marine and freshwater animals, and wild animals.

As used herein, the term “pharmaceutically acceptable salt” refers to any salt (e.g., obtained by reaction with an acid or a base) of a compound of the present invention (e.g., comprising a viable yeast cell or cell wall component of the invention) that is physiologically tolerated in the target subject (e.g., a mammalian, humans, avian, bovine, porcine, equine, ovine, caprine, canine, feline, piscine, camelid, rodent species as well as fish and shellfish subjects subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metal (e.g., sodium) hydroxides, alkaline earth metal (e.g., magnesium) hydroxides, ammonia, and compounds of formula NW₄₊, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄₊, and NW₄₊ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic and/or prophylactic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “kit” refers to a packaged set of materials.

As used herein “anti-adherence modulators” and/or “anti-adhesion modulators” refer to modulators that block adherence (e.g., block a compound from adhering to fimbria, and/or block adherence of bacteria to mucous epithelia cells and/or to other types of cells).

### DETAILED DESCRIPTION OF THE INVENTION

Radioactive binding assays have been shown to measure bacterial adherence to intestinal mucus, and that certain agents effectively prevent such adherence (see, e.g., Conway, et al., 1990, Infection and Immunity 58:3178-3182; herein incorporated by reference in its entirety). In particular, radioactive binding assays shown to measure bacterial adherence to intestinal mucus have further shown the effect of Bio-Mos, a mannoprotein derived from the cell wall of Saccharomyces cerevisiae, on inhibiting bacterial adherence. However, no non-radioactive routine method is available for detecting, identifying, and measuring bacterial adherence to mucus. The methods and compositions of the present invention overcome such limitations through providing non-radioactive methods for detecting, identifying, and measuring bacterial adherence to mucus.

In particular, the present invention provides a simple and accurate immunoassay for measuring bacterial adherence to mucus and for testing the effect of products that modulate (e.g., inhibit, promote) adherence. In some embodiments, the immunoassay is a Western blot. In some embodiments, the immunoassay is a radio-immunoassay. In some embodiments, the immunoassay is an immunofluorescence assay. In some embodiments, the immunoassay is an ELISA based assay. An ELISA based method is an attractive alternative to a radioactive assay due to flexibility in the use of difference combinations of primary and secondary antibodies and various colorimetric detection systems for different microbial species. Accordingly, the present invention provides ELISA based methods for detecting and identifying, bacterial adherence to mucus (e.g., intestinal mucosal lining).

Thus, in some embodiments, the present invention provides a non-radioactive, colorimetric assay for monitoring and/or characterizing interaction (e.g., binding, attachment, affinity, etc.) between mucus and bacterial cells. In some embodiments, the non-radioactive assay is as sensitive and/or more sensitive than a radioactive assay utilized for similar monitoring and/or characterizing. In some embodiments, a non-radioactive, colorimetric assay of the invention is utilized to monitor and/or characterize the ability of one or more test agents to alter (e.g., inhibit and/or enhance) bacterial cell interaction (e.g., binding, attachment, affinity, etc.) with mucus.

For example, in some embodiments, the present invention provides an enzyme linked immunosorbant as assay (ELISA) method for monitoring and/or characterizing interaction (e.g., binding, attachment, affinity, etc.) between mucus and bacterial cells (e.g., as described in Examples 1-16. In some embodiments, the assay is performed at room temperature. In some embodiments, the assay is performed at 37° C. In some embodiments, the assay is optimized as described in Examples 2-15. In some embodiments, plates (e.g., microtitre plates (e.g., MAXISORP plates (e.g., containing 6, 12, 24, 48, 96, 128 or more wells))) are coated with mucus. The present invention is not limited by the type, source or amount of mucus. In some embodiments, the mucus utilized is animal mucus. In some embodiments, mucus is obtains from a pig, a chicken, a cow, an equine, a canine, a feline, or other type of animal. In some embodiments, the mucus is obtained from one or more portions of the digestive tract. For example, in some embodiments, mucus is obtained from the ileum (e.g., proximal ileum, distal ileum, etc.), duodenum, caecum, colon and/or other part of the digestive tract. The present invention is not limited by the amount of mucus utilized to coat the plates (e.g., depending upon the number and/or size of the wells on the plate). In some embodiments, mucus is diluted in a coating buffer and then utilized for coating the plates. In some embodiments, the coating buffer is a solution comprising 1 liter of water into which 1.6 g Na₂C0₃, 2.94 g NaHC0₃, and 0.2 g Na-azide have been dissolved and the pH adjusted to 9.6, or similar buffer. In some embodiments, a coating buffer comprising between about 0.001-0.2 mg of mucus protein per ml of coating buffer is utilized to coat each well on the plate, although greater (e.g., 0.3 mg/ml, 0.4 mg/ml, 0.5 mg/ml, 0.75 mg/ml, 1.0 mg/ml or more) or lesser (e.g., 0.0005 mg/ml or less) amounts may be utilized. In some embodiments, about 300 μl of the coating suspension is utilized to coat each well, although greater (e.g., 400 μl, 500 μl, 600 μl , 700 μl or more) or lesser (e.g., 200 μl, 100 μl, 50 μl, 25 μl or less) volumes of coating solution may be utilized (e.g., depending upon the size of the well, amount of signal desired, or other factors (e.g., bacterial adherence)). Once the coating solution is added to the wells, the mucus is allowed to coat each well for a period of time (e.g., about 1 hour, about 2 hours, about 3 hours, about 6 hours, about 12 hours, about 24 hours, or more) at a constant temperature (e.g., 4° C., room temperature, or warmer (e.g., 37° C.)). In some embodiments, the plates are covered during incubation (e.g., to prevent evaporation of the coating solution). Sometime during the coating period, a test agent (e.g., that is to be tested for its ability to alter (e.g., inhibit and/or enhance) bacterial binding to the mucus) is prepared. The test agent is diluted in any appropriate buffer (e.g., phosphate buffered saline (PBS) (e.g., a PBS solution generated by dissolving 8.0 g NaCl, 0.2 g KCl, 1.4 g Na₂HP0₄×2H₂0, 0.2 g KH₂P0₄ into 1 of water and adjusting to pH 7.4). The present invention is not limited by the type of test agent. Indeed, a variety of test agents can by monitored and/or characterized utilizing methods of the invention including, but not limited to, those described herein.

After coating is complete, the coating solution is removed from the wells (e.g., without mixing the contents of the wells) and each well is washed with an appropriate volume (e.g., 100 ml, 200 ml, 300 ml, 400 ml or more) of washing solution (e.g., PBS).

Bacteria to be monitored and/or characterized for interaction with mucus are prepared by collecting the bacteria under conditions that do not disrupt the integrity of the bacteria. The present invention is not limited to any particular type of bacteria nor to any particular growth phase of the bacteria. Indeed, a variety of bacteria may be monitored and/or characterized in an assay of the invention including but not limited to the types of bacteria described herein. Once collected (e.g., via centrifuging to pellet the bacterial cells), the bacteria are resuspended in buffer (e.g., PBS) to a desired concentration depending upon how many bacteria are desired per well. In some embodiments, the number of bacteria added per well is about 10⁷, although greater (e.g., 10⁸,10⁹, 10¹⁰) or fewer (e.g., 10⁶,10⁵, 10⁴) bacteria may be added to each well. After the final wash of the plates, bacteria are added to the wells. In some embodiments, a test agent solution is added to the bacteria suspension just prior to adding to the wells. The amount test agent and the amount of cells can be varied as described herein. Once added to the wells, the bacteria and/or bacteria plus test agent are allowed to incubate in the wells for a set period of time (e.g., 1 hour, 2 hours, 4 hours, 8 hours or more). Post incubation, the wells are washed (e.g., one, two, three or more times with PBS). Post washing, a blocking buffer (e.g., fetal bovine serum (FBS), bovine serum albumin (BSA), milk, or other suitable blocking agent (e.g., 10% FBS diluted in PBS) is added to each well (e.g., using the same volume of blocking buffer that was utilized to coat the wells with mucus). The blocking solution is incubated in the wells for a set period of time (e.g., about 1 hour, about 2 hours, about 3 hours or more) at a constant temperature (e.g., 4° C., room temperature, or warmer (e.g., 37° C.)). Blocking buffer is removed and then a primary antibody (e.g., with specific affinity for the bacteria being monitored and/or characterized) is added to the wells. The primary antibody is diluted (e.g., at 1:500, 1:1000, 1:2500, 1:5000 or more) in the blocking buffer. The volume of diluted primary antibody to be added to the wells is about 100 ml to about 400 ml (e.g., 200 ml) and is incubated in the wells for a set period of time (e.g., about 1 hour, about 2 hours, about 3 hours or more) at a constant temperature (e.g., 4° C., room temperature, or warmer (e.g., 37° C.)). The present invention is not limited to by the primary antibody utilized. Indeed, any antibody with specific affinity for the type of bacteria being monitored and/or characterized may be utilized. In some embodiments, the primary antibody is a polyclonal antibody. In some embodiments, the primary antibody is a monoclonal antibody. In some embodiments, the primary antibody is an antibody fragment. In some embodiments, the primary antibody is a conjugated antibody. For example, in some embodiments, the primary antibody is biotin conjugated. In some embodiments, the primary antibody is a biotin conjugated polyclonal antibody to E. coli. Post primary antibody incubation, the wells are washed (e.g., one, two, three or more times) using a washing buffer (e.g., PBS). Washing buffer is removed and then a secondary antibody (e.g., with specific affinity for the primary antibody is added to the wells. The secondary antibody is also diluted (e.g., at 1:500, 1:1000, 1:2500, 1:5000 or more) in the blocking buffer. The present invention is not limited to the type of secondary antibody utilized. In some embodiments, the secondary antibody is a polyclonal antibody. In some embodiments, the secondary antibody is a monoclonal antibody. In some embodiments, the secondary antibody is an antibody fragment. In some embodiments, the secondary antibody is a conjugated antibody. In some embodiments, the secondary antibody is conjugated to streptavidin. In some embodiments, the secondary antibody is conjugated to an enzyme (e.g., peroxidase, phosphatase, etc.). The volume of diluted secondary antibody to be added to the wells is about 100 ml to about 400 ml (e.g., 200 ml) and is incubated in the wells for a set period of time (e.g., about 1 hour, about 2 hours, about 3 hours or more) at a constant temperature (e.g., 4° C., room temperature, or warmer (e.g., 37° C.)). After the incubation, the wells are washed (e.g., two, three, four, five or more times) with a washing buffer (e.g., PBS). Post the last wash, a colorimetric substrate is added to the wells. The present invention is not limited by the type of substrate utilized. Exemplary substrates include, but are not limited to, 3.3′,5.5′-tetramethylbenzidine (TMB) (e.g., for peroxidase-conjugated secondary antibodies), (p-NitroPhenyl Phosphate (pNPP) (e.g., for phosphates conjugated antibodies), etc.). Color develops in the wells and is detected and/or quantified (e.g., using a spectrophotometer). Color development can be stopped by the addition of an acidic buffer (e.g., 2M H₂S0₄) at any time point (e.g., to prevent strong color signal production (e.g., in order to quantify bacterial attachment)).

The present invention is not limited to a particular ELISA based method for detecting, identifying, and measuring bacterial adherence to mucus. In some embodiments, methods are provided wherein 1) plates configured for use in ELISA based assays are coated with a mucus sample, 2) bacteria are applied to the mucus coated plates, 3) primary antibodies directed to bacteria are applied, 4) secondary antibodies directed to the primary antibodies are applied, 5) a liquid substrate is applied, and 6) bacterial adherence is measured. In some embodiments, washing steps are applied between one or more of the steps. In some embodiments, blocking solution is applied between one or more of the steps. The methods are not limited to particular types or kinds of mucus samples, bacteria, primary antibodies, secondary antibodies, liquid substrate, and/or techniques for measuring bacterial adherence.

The methods for detecting, identifying, and measuring bacterial adherence to mucus is not limited to a particular type of bacteria. Indeed, any type of bacteria may be used in the present invention. Examples of bacteria include, but are not limited to, Acidobacteria, Actinobacteria, Aquificae, Bacteroidetes/Chlorobi, Chlamydiae/Verrucomicrobia, Chloroflexi, Chrysiogenetes, Cyanobacteria, Deferribacteres, Deinococcus-Thermus, Dictyoglomi, Fibrobacteres, Firmicutes, Fusobacteria, Gemmatimonadetes, Nitrospirae, Planctomycetes, Proteobacteria, Spirochaetes, Synergistetes, Tenericutes, Thermodesulfobacteria, and Thermotogae. In some embodiments, the bacteria is pathogenic bacteria such as, for example, Bordetella (e.g., Bordetella pertussis), Borrelia (e.g., Borrelia burgdorferi), Brucella (e.g., Brucella abortus, Brucella canis, Brucella melitensis, Brucella suis), Campylobacter (e.g., Campylobacter jejuni), Chlamydia (e.g., Chlamydia pneumoniae, Chlamydia psittaci, Chlamydia trachomatis), Clostridium (e.g., Clostridium botulinum, Clostridium difficile, Clostridium perfringens, Clostridium tetani), Corynebacterium (e.g., Corynebacterium diphtheriae), Enterococcus (e.g., Enterococcus faecalis, Enterococcus faecum), Escherichia (e.g., Escherichia coli), Francisella (e.g., Francisella tularensis), Haemophilus (e.g., Haemophilus influenzae), Helicobacter (e.g., Helicobacter pylori), Legionella (e.g., Legionella pneumophila), Leptospira (e.g., Leptospira interrogans), Listeria (e.g., Listeria monocytogenes), Mycobacterium (e.g., Mycobacterium leprae, Mycobacterium tuberculosis), Mycoplasma (e.g., Mycoplasma pneumoniae), Neisseria (e.g., Neisseria gonorrhoeae, Neisseria meningitidis), Pseudomonas (e.g., Pseudomonas aeruginosa), Rickettsia (e.g., Rickettsia rickettsii), Salmonella (e.g., Salmonella typhi, Salmonella typhimurium), Shigella (e.g., Shigella sonnei), Staphylococcus (e.g., Staphylococcus aureus, Staphylococcus epidermidis, Staphylococcus saprophyticus), Streptococcus (e.g., Streptococcus agalactiae, Streptococcus pneumoniae, Streptococcus pyogenes), Treponema (e.g., Treponema pallidum), Vibrio (e.g., Vibrio cholerae), and Yersinia (e.g., Yersinia pestis). In some embodiments, the bacteria are selected from particular strains of E. Coli known to have strong adherence to pig mucus (e.g., E. coli ALI84 and/or ALI446).

The present invention is not limited to a particular manner of preparing and/or utilizing bacteria within the ELISA based methods for detecting, identifying, and measuring bacterial adherence to mucus. In some embodiments, the bacteria are inactivated prior to its use (e.g., for storage purposes) and activated during testing. The methods are not limited to a particular method for inactivating the bacteria. Examples of inactivating the bacteria include, but are not limited to, freezing the bacteria, suspending the bacteria with ethanol, suspending the bacteria with glutaraldehyde, suspending the bacteria with formalin, irradiating the bacteria with ultraviolet irradiation, suspending the bacteria with dimethyl sulfoxide, and heating the bacteria before cooling for storage. The methods are not limited to a particular manner of activating the inactivated bacteria for testing purposes. In some embodiments, the bacteria are activated (e.g., harvested) through centrifugation techniques.

The methods are not limited to a particular manner of inactivating bacteria with ethanol. In some embodiments, the bacteria are grown and transferred in a fresh medium (e.g., 10% inoculums) prior to inactivation (e.g., one day prior to inactivation) with ethanol. Next, the bacteria are inactivated and preserved by adding ethanol directly to the bacterial culture (e.g., to approximately a final concentration of 40% vol/vol (e.g., 20% vol/vol; 30% vol/vol; 33% vol/vol; 35% vol/vol; 37% vol/vol; 40% vol/vol; 42% vol/vol; 45% vol/vol; 50% vol/vol; 60% vol/vol). In some embodiments, bacteria inactivated with ethanol (e.g., to a final concentration of approximately 40% vol/vol) are stored at approximately +4° C. (e.g., 2° C.; 3° C.; 4° C.; 5° C.; 6° C.). In some embodiment, bacteria inactivated with ethanol (e.g., to a final concentration of approximately 40% vol/vol) (e.g., stored at approximately +4° C.) is activated (e.g., harvested) through centrifugation. In some embodiments, the bacteria are suspended in phosphate buffer saline (e.g., PBS) (e.g., 8.0 g NaCl, 0.2 g KCl, 1.4 g Na₂HPO₄×2H₂O, 0.2 g KH₂PO₄, ad. 1000 ml MilliQ H₂O, pH 7.4).

The methods for detecting, identifying, and measuring bacterial adherence to mucus is not limited to a particular type of mucus. Indeed, any type of mucus may be used in the present invention. In some embodiments, the mucus used is from a pig (e.g., pig colon mucus) (e.g., pig intestine mucus (e.g., scraped from the proximal ileum of an approximately one year old pig)).

The present invention is not limited to a particular manner of preparing and/or utilizing mucus samples (e.g., mucus from a pig) within the ELISA based methods for detecting, identifying, and measuring bacterial adherence to mucus. In some embodiments, the mucus samples are suspended with a coating buffer. The methods are not limited to a particular configuration for the coating buffer. In some embodiments, the coating buffer comprises 1.6 g Na₂CO₃ (dry), 2.94 g NaHCO3, 0.2 g Na-azide, 11H₂0 with pH 9.6 (by mixing the components ends up to be 9.7).

In some embodiments, the mucus samples are coated onto plates (e.g., wells) (e.g., 96 well plates) (e.g., 96 well MaxiSorp plates) configured for use in ELISA based assays. The mucus samples are not limited to a particular manner of coating onto plates configured for use in ELISA based assays. In some embodiments, the mucus samples are directly coated onto the plates just prior to the testing. In some embodiments, the mucus samples are pre-coated onto the plates so as to permit long term storage prior to testing. The methods are not limited to particular methods of pre-coating plates configured for use in ELISA based assays with mucus samples (e.g., mucus from pig intestine). In some embodiments, pre-coating plates configured for use in ELISA based assays with mucus samples is accomplished through coating the plates with the mucus samples and subsequently freezing the coated plates. In some embodiments, pre-coating plates configured for use in ELISA based assays with mucus samples is accomplished through coating the plates with the mucus samples and subsequently air-drying the coated plates. The methods are not limited to a particular manner of re-hydrating mucus samples pre-coated onto plates configured for use in ELISA based assays. In some embodiments, re-hydration is accomplished through exposing the samples to phosphate buffer saline (e.g., PBS) (e.g., 8.0 g NaCl, 0.2 g KCl, 1.4 g Na₂HPO₄×2H₂O, 0.2 g KH₂PO₄, ad. 1000 ml MilliQ H₂O, pH 7.4).

The methods for detecting, identifying, and measuring bacterial adherence to mucus is not limited to a particular type of primary antibody. In some embodiments, the primary antibodies are directed toward the bacteria for which mucosal adherence is being tested. In some embodiments, the primary antibody is an HRP-conjugated polyclonal antibody to E. coli O and K antigenic serotypes (Acris catalogue number BP2022HRP). In some embodiments, the primary antibody is a polyclonal antibody to E. coli O and K antigenic serotypes (Acris catalogue number BP2022). In some embodiments, the primary antibody is a biotin-conjugated polyclonal antibody to E. coli O and K antigenic serotypes (Acris catalogue number BP1021B).

The methods for detecting, identifying, and measuring bacterial adherence to mucus is not limited to a particular type of secondary antibody. In some embodiments, the secondary antibodies are configured for detecting binding of the primary antibody with bacteria bound to mucus. As such, in some embodiments, the secondary antibodies are directed toward the primary antibody. In some embodiments, the secondary antibody is an affinity purified Rabbit anti-Goat IgG-HRP (Acris catalogue number R1317HRP). In some embodiments, the secondary antibody is an affinity purified Rabbit anti-Goat IgG-AP (Acris catalogue number R1317AP). In some embodiments, the secondary antibody is a polyclonal FITC-conjugated antibody to Goat IgG (H&L) (Acris catalogue number R13 17F). In some embodiments, the secondary antibody is Streptavidin-Alkaline Phosphatase from Streptomyces avidinii (Sigma catalogue number S2890). In some embodiments, the secondary antibody is Streptavidin-Peroxidase from Streptomyces avidinii (Sigma catalogue number S5512).

In some embodiments, the primary antibodies and secondary antibodies are diluted in a blocking solution. The methods are not limited to a particular type of blocking solution. In some embodiments, the blocking solution is milk. In some embodiments, the blocking solution is fetal bovine serum (FBS). In some embodiments, the blocking solution is bovine serum albumin (BSA).

The methods for detecting, identifying, and measuring bacterial adherence to mucus is not limited to a particular type of liquid substrate. In some embodiments, the liquid substrate is configured to facilitate detection of the binding of the primary antibody and/or the secondary antibody within the assay 3,3′,5,5′ -tetramethylbenzidine (TMB) (Sigma catalogue number T4319). In some embodiments, the liquid substrate is TMB slow kinetic form (later TMB slow) (Sigma catalogue number T0440). In some embodiments, the liquid substrate is TMB super sensitive (later TBM super) (Sigma catalogue number T4444). In some embodiments, the liquid substrate is P-nitrophenyl phosphate (Sigma catalogue number N7653). In some embodiments, the liquid substrate is 2,2′-Azino-bis(3-ethylbenzothiazoline-6-sulfonic acid) (AzBTS; Sigma catalogue number A3219)+ABTS microwell enhancer (Sigma catalogue number AI227).

The methods for detecting, identifying, and measuring bacterial adherence to mucus is not limited to a particular technique for measuring such bacterial adherence to mucus. In some embodiments, bacterial adherence to mucus is measured visually (e.g., using imagery and/or photography). In some embodiments, bacterial adherence to mucus is measured with an ELISA plate reader. In some embodiments, the technique employed to measure bacterial adherence to mucus detects, measures and quantifies, for example, absorbance, fluorescence intensity, luminescence, time-resolved fluorescence, and/or fluorescence polarization.

In some embodiments, the methods for detecting, identifying, and measuring bacterial adherence to mucus (e.g., the ELISA based methods) are used to identify agents that modulate bacterial adherence to mucus. In some embodiments, methods for detecting, identifying and/or measuring bacterial adherence of the invention are utilized to generate and/or identify optimized (e.g., second, third, fourth or more generation) compositions (e.g., that show greater efficacy (e.g., at preventing bacterial adherence) than an earlier generation composition. The methods are not limited to a particular technique for identifying agents that modulate bacterial adherence to mucus and/or cells (e.g., epithelial cells). In some embodiments, a potential modulator of bacterial adherence to mucus and/or cells (e.g., epithelial cells) is co-applied with a bacterial sample to a plate coated with mucus and/or cells (e.g., epithelial cells) (e.g., pig intestine mucus and/or epithelial cells), and primary and secondary antibodies, and liquid substrate subsequently applied. In some embodiments, characterization of the modulation activity of the agent is accomplished through comparing bacterial adherence in the presence and absence of the agent. For example, agents that increase bacterial adherence to mucus and/or cells (e.g., epithelial cells) are characterized as, for example, facilitators of adherence between that specific type of bacteria and that specific type of mucus and/or cells (e.g., epithelial cells). Agents that decrease bacterial adherence to mucus and/or cells (e.g., epithelial cells)are characterized as, for example, inhibitors of adherence between that specific type of bacteria and that specific type of mucus and/or cells (e.g., epithelial cells). The methods are not limited to a particular type or kind of potential agent. In some embodiments, the agent is, for example, a naturally occurring molecule, a synthetically derived molecule, or a recombinantly derived molecule.

In some embodiments, the methods involve pre-application of one or more agents known to modulate bacterial adherence to mucus or epithelial cells as a prophylactic, or preventative measure. For example, in some embodiments, the methods involve pre-application of one or more agents known to inhibit bacterial adherence to mucus. Methods of the invention are not limited to any particular type of agent known to inhibit bacterial adherence to mucus and/or cells (e.g., epithelial cells). For example, in some embodiments, the agent known to inhibit bacterial adherence to mucus is Bio-Mos (e.g., a mannoprotein derived from the cell wall of Saccharomyces cerevisiae), although the present invention is not so limited. In some embodiments, the agent known to inhibit bacterial adherence to mucus is identified through use of the ELISA based methods of the present invention. In some embodiments, the methods involve co-application of one or more agents known to enhance bacterial adherence to mucus. The methods are not limited to a particular type of agent known to enhance bacterial adherence to mucus. In some embodiments, the agent known to enhance bacterial adherence to mucus is identified through use of the ELISA based methods of the present invention.

In some embodiments, the methods involve co-application of one or more agents known to modulate bacterial adherence to mucus. For example, in some embodiments, the methods involve co-application of one or more agents known to inhibit bacterial adherence to mucus. The methods are not limited to a particular type of agent known to inhibit bacterial adherence to mucus. In some embodiments, the agent known to inhibit bacterial adherence to mucus is Bio-Mos (e.g., a mannoprotein derived from the cell wall of Saccharomyces cerevisiae). In some embodiments, the agent known to inhibit bacterial adherence to mucus is identified through use of the ELISA based methods of the present invention. In some embodiments, the methods involve co-application of one or more agents known to enhance bacterial adherence to mucus. The methods are not limited to a particular type of agent known to enhance bacterial adherence to mucus. In some embodiments, the agent known to enhance bacterial adherence to mucus is identified through use of the ELISA based methods of the present invention.

In some embodiments, the methods involve post-application of one or more agents known to modulate bacterial adherence to mucus or epithelial cells after infectious disease has been removed. For example, in some embodiments, the methods involve post-application of one or more agents known to inhibit bacterial adherence to mucus and/or cells (e.g., epithelial cells). The methods are not limited to a particular type of agent known to inhibit bacterial adherence to mucus. In some embodiments, the agent known to inhibit bacterial adherence to mucus is Bio-Mos (e.g., a mannoprotein derived from the cell wall of Saccharomyces cerevisiae). In some embodiments, the agent known to inhibit bacterial adherence to mucus is identified through use of the ELISA based methods of the present invention. In some embodiments, the methods involve co-application of one or more agents known to enhance bacterial adherence to mucus. The methods are not limited to a particular type of agent known to enhance bacterial adherence to mucus. In some embodiments, the agent known to enhance bacterial adherence to mucus is identified through use of the ELISA based methods of the present invention.

In some embodiments, assays of the invention are utilized to identify and/or characterize anti-adherence compounds for intestinal and/or urinary tract bacteria (e.g., bacteria that colonize mucosal surfaces of the intestinal and/or urinary tract). In some embodiments, anti-adherence compounds are identified that are utilized to prevent and/or treat disease and/or signs and/or symptoms of the same (e.g., salmonellosis, metritis, etc. (e.g., in animals (e.g., reproductive animals such as dairy cows, sows, etc.))). In some embodiments, assays of the invention can be performed anywhere a microplate reader can be utilized including, but not limited to, in a lab (e.g., university, private, public, or other type of lab), in the field (e.g., on a ranch, a farm, or site of user of the assay), etc. In some embodiments, assays and/or assay components are sold commercially and utilized by an end user (e.g., purchaser of an assay) in the end user's own lab (e.g., to check product (e.g., anti-adherence compound) efficacy, performance and/or consistency). Thus, the present invention provides compositions and methods that allow users of an assay to perform their own quality characterization of compounds (e.g., anti-adherence compounds) at a user's site (e.g., on site at use of anti-adherence compound (e.g., BIO-MOS). In some embodiments, information (e.g., efficacy, quality, consistency, etc.) related to the anti-adherence compound generated using assays of the invention is collected. In some embodiment, information is collected using a database (e.g., online database) or mailings. In some embodiments, the information and/or data collected related to anti-adherence compound (e.g., efficacy, quality, consistency, etc.) is utilized in a quality control program. In some embodiments, the information and/or data collected related to anti-adherence compound (e.g., efficacy, quality, consistency, etc.) is utilized by a provider and/or manufacturer of the anti-adherence compound to monitor activity of the compound. In some embodiments, information and/or data collected related to anti-adherence compound (e.g., efficacy, quality, consistency, etc.) is utilized with animal health data collected at the site of use of the anti-adherence compound (e.g., to provide information related animal performance),In some embodiments, a preferred physical form of an agent identified through use of the ELISA based methods of the present invention (e.g., identified as a modulator of bacterial adherence to mucus) is a dry free-flowing powder suitable for direct inclusion into animal feeds or as a direct supplement to an animal. In other embodiments, a preferred physical form of an agent identified through use of the ELISA based methods of the present invention (e.g., identified as a modulator of bacterial adherence to mucus) is a liquid or a paste that is administered post-pellet or through drinking water.

Compositions of the invention comprising an agent identified through use of the ELISA based methods of the present invention (e.g., identified as a modulator of bacterial adherence to mucus) can be added to any commercially available feedstuffs for livestock, companion animals, fishes, and shellfishes including, but not limited to, Total Mixed Ration (TMR), forage(s), pellet(s), concentrate(s), premix(es) coproduct(s), grain(s), distiller grain(s), molasses, fiber(s), fodder(s), grass(es), hay, kernel(s), leaves, meal, soluble(s), and supplement(s) Compositions of the invention comprising an agent identified through use of the ELISA based methods of the present invention (e.g., identified as a modulator of bacterial adherence to mucus) are incorporated directly into animal feeds (e.g., commercially available pelleted feeds). When incorporated directly into animal feeds, compositions comprising an agent identified through use of the ELISA based methods of the present invention may be added to the animal, fish, or shellfish feedstuffs in amounts ranging from about 0.0125% to about 0.4% by weight of feed. In some embodiments, the composition is added to animal, fish, shellfish feedstuffs in amounts from about 0.025% to about 0.2% by weight of feed. Alternatively, compositions of the invention are directly fed to animals as a supplement (e.g., in an amount ranging from about 2.5 to about 20 grams per animal per day). One of ordinary skill in the art immediately appreciates that the amount of a composition fed varies depending upon animal species, size, and the type of feedstuff to which a composition of the invention is added.

Compositions of the invention comprising an agent identified through use of the ELISA based methods of the present invention (e.g., identified as a modulator of bacterial adherence to mucus) can be fed to any animal, including but not limited to ruminant and equine species. When admixed with feed or fed as a supplement, compositions of the invention comprising an agent identified through use of the ELISA based methods of the present invention (e.g., identified as a modulator of bacterial adherence to mucus) modulate (e.g., increase or decrease depending on the agent) bacterial adherence to mucus in the animal, improving performance and health and reducing incidence of disease.

In some embodiments, the present invention provides methods for treating disorders caused by pathogenic bacteria through administering to a subject an agent known to modulate (e.g., inhibit, facilitate) bacterial adherence to mucus. In some embodiments, the agent is identified through use of the ELISA based methods of the present invention. In some embodiments, the disorder is caused by Bacillus anthracis (e.g., cutaneous anthrax, pulmonary anthrax, gastrointestinal anthrax), and in some embodiments the method involves co-administration of penicillin, doxycycline and/or ciprofloxacin. In some embodiments, the disorder is caused by Bordetella pertussis (e.g., whooping cough, secondary bacterial pneumonia), and in some embodiments the method involves co-administration of macrolide antibiotics (e.g., azithromycin, erythromycin, clarithromycin). In some embodiments, the disorder is caused by Borrelia burgdorferi (e.g., lyme disease), and in some embodiments the method involves co-administration of cephalosporins, amoxicillin, and/or doxycycline. In some embodiments, the disorder is caused by Brucella pathogenic bacteria (e.g., Brucella abortus, Brucella canis, Brucella melitensis, Brucella suis) (e.g., brucellosis), and in some embodiments the method involves co-administration of doxycycline, streptomycin, and/or gentamycin. In some embodiments, the disorder is caused by Campylobacter jejuni (e.g., acute enteritis), and in some embodiments the method involves co-administration of ciprofloxacin. In some embodiments, the disorder is caused by Chlamydia pneumoniae (e.g., community-acquired respiratory infection), and in some embodiments the method involves co-administration of doxycycline, and/or erythromycin. In some embodiments, the disorder is caused by Chlamydia psittaci (e.g., Psittacosis), and in some embodiments the method involves co-administration of tetracycline, doxycycline, and/or erythromycin. In some embodiments, the disorder is caused by Chlamydia trachomatis (e.g., nongonococcal urethritis (NGU), trachoma, inclusion conjunctivitis of the newborn (ICN), lymphogranuloma venereum (LGV)), and in some embodiments the method involves co-administration of azithromycin, erythromycin, tetracyclines, and/or doxycycline. In some embodiments, the disorder is caused by Clostridium botulinum (e.g., botulism), Clostridium difficile, Clostridium perfringens, clostridium tetani (e.g., tetanus), Corynebacterium diphteriae (e.g., diphtheria), Enterococcus faecalis, Enterococcus faecum, Escherichia coli, Francisella tularensis (e.g., tularemia), Haemophilus influenzae, Helicobacter pylori, Legionella pneumophila (e.g., Legionnaire's Disease), Leptospira interrogans, Listeria monocytogenes, Mycobacterium leprae (e.g., Hansen's disease), Mycobacterium tuberculosis (e.g., tuberculosis), Mycoplasma pneumoniae, Neisseria gonorrhoeae, Neisseria meningitidis, Pseudomonas aeruginosa, Rickettsia rickettsii, Salmonella typhi, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis (e.g., plague).

In some embodiments, the present invention provides kits configured to permit a user to practice the methods of the present invention (e.g., methods for detecting, identifying, and measuring bacterial adherence to mucus). In some embodiments, the kits contain one or more the following ingredients, mucus samples, plates coated with mucus samples, bacteria, primary antibodies, secondary antibodies, liquid substrate, washing solutions, a device configured to interpret ELISA based assays, instructions, an agent known to decrease bacterial adherence to mucus (e.g., Bio-Mos) (e.g., an agent identified through use of the ELISA based methods of the present invention), an agent known to increase bacterial adherence to mucus (e.g., an agent identified through use of the ELISA based methods of the present invention), and additional treatment agents (e.g., antibiotics).

### Experimental

The following examples are provided in order to demonstrate and further illustrate certain preferred embodiments and aspects of the present invention and are not to be construed as limiting the scope thereof.

### Example 1

### Materials and Methods Utilized in Colorimetric ELISA Versus Radioactive Detection of Bacterial Adherence to Intestinal Matter

Bacterial strains. Two E. coli strains were selected for the development project: E. coli ALI84 and ALI446. The former was originally isolated from sick birds and the latter strain was isolated from a pig with diarrhea. These strains were selected because they have displayed adherence to pig mucus. The bacteria were grown in LB-broth and transferred to a fresh medium (culture:medium 1:10) on the day before experiments. The number of bacteria was estimated according to culturing time.

Antibodies. Antibodies were purchased from Acris Antibodies GmbH, Germany and Sigma Aldrich, Germany. Primary antibodies included: a HRP-conjugated polyclonal antibody to E. coli O and K antigenic serotypes (Acris catalogue number BP2022HRP); a polyclonal antibody to E. coli O and K antigenic serotypes (Acris catalogue number BP2022); and a biotin-conjugated polyclonal antibody to E. coli 0 and K antigenic serotypes (Acris catalogue number BP1021B).

Secondary antibodies include: an affinity purified Rabbit anti-Goat IgG-HRP (Acris catalogue number R1317HRP); an affinity purified Rabbit anti-Goat IgG-AP (Acris catalogue number R1317AP); a polyclonal FITC-conjugated antibody to Goat IgG (H&L) (Acris catalogue number R13 17F); Streptavidin-Alkaline Phosphatase from Streptomyces avidinii (Sigma catalogue number S2890); Streptavidin-Peroxidase from Streptomyces avidinii (Sigma catalogue number S5512). ELISA-substrates were purchased as ready-to-use solutions from Sigma-Aldrich, Germany: 3,3′,5,5′ -tetramethylbenzidine (TMB) (Sigma catalogue number T4319); TMB slow kinetic form (later TMB slow) (Sigma catalogue number T0440); TMB super sensitive (later TBM super) (Sigma catalogue number T4444); P-nitrophenyl phosphate (Sigma catalogue number N7653); 2,2′-Azino-bis(3-ethylbenzothiazoline-6-sulfonic acid) (AzBTS; Sigma catalogue number A3219)+ABTS microwell enhancer (Sigma catalogue number A1227).

Buffers. Originally, HEPES-Hanks buffer (pH 7.4) was used for washing the wells except for the final wash in which PBS was used to avoid disturbance of the red colored HEPES-Hanks in ELISA. In some embodiments, PBS (phosphate buffered saline, pH 7.4) was included in all steps in place of HEPES-Hanks buffer.

Plates. For ELISA experiments, 96-well immuno plates were used (MaxiSorp, Nunc, Denmark, later in this report “Maxisorp plates”). For scintillation experiments, polyethylene terephthalate microtiter plates were used (96-well PET sample plate, 1450-401; Wallac Oy, Turku; referred to herein as “soft plates”) for use in a scintillation counter.

A conventional radioactive binding/attachment assay, described below, was utilized as a control for the colorimetric ELISA.

Radioactive labeling of bacteria for radioactive attachment assay. The bacteria were grown overnight at +37° C. and the bacterial suspension diluted 1:10 into a new batch of LB and methyl-1,-2, 3H thymidine (117 I-lCi/mmol; Amersham) was added. The bacteria were incubated for 2 h at +37° C. and collected by centrifugation for 5 min at 3000 g. Bacterial pellet was resuspended in HEPES-Hanks buffer or PBS and used in the attachment assay.

Radioactive attachment assay. 200 ul of diluted bacterial suspension was added in microtiter wells and the plates were incubated for 1 h at +37° C. Unbound cells were removed by washing three times with 300 μl of HEPES-Hanks buffer or PBS. 250 ul of scintillation liquid was added and the radioactivity measured with a scintillation counter.

Mucus isolation and immobilization. The plates were coated with different concentrations of mucus from different animals. Mucus from the proximal ileum of a −1 year old pig was used unless otherwise indicated. The mucus was scraped from the surface of intestine and washed. Crude mucus extract was stored at −80° C. until usage. For coating, the protein concentration of the mucus was adjusted to 0.0-0.2 mg/ml using sodium carbonate buffer (coating buffer, pH 9.6), and the optimal mucus concentration for bacterial adherence tested. Mucus solution was immobilized on the plates by introducing 300 μl or 200 μl of mucus into each 350 ul well. The plate was then incubated at +4° C. overnight. Extra mucus was removed from the microtiter wells by washing twice with 300 μl of HEPES-Hanks buffer or PBS.

Microfuge method used in testing unspecific color development by E. coli with ELISA-substrates. A fresh culture of E. coli (˜10⁸ bacteria/ml) was divided into microfuge tubes (˜10⁷/tube). The tubes were centrifuged for 5 minutes/3000 g, and the supematant was removed. The bacteria were suspended to 700 μl of ELISA substrate. The absorbance of the suspension was read with spectrophotometer at 370, 405 and 630 nm after five minutes and then each 15 minutes until 3 hours.

FITC method used in testing primary antibody specificity. The specificity of the primary antibody (polyclonal anti-E. coli, BP2022, compatible with FITC-conjugated secondary antibody) was tested with fluorescence microscopy. 10⁸ bacteria were introduced to each microfuge tube and washed three times with 1 ml of PBS (and centrifuged 3000 g/5 min between the washes). Primary antibody diluted to 1:100, 1:500, 1:1000 in 1% BSA/PBS was introduced to each tube and incubated at room temperature for 45 min. 1% BSA/PBS was used as a negative control. The bacteria were washed three times with PBS and the secondary antibody (FITC-conjugated rabbit antigoat IgG) was introduced in dilutions of 1:100,1:500, and 1:1000 in 1% BSA1PBS. The tubes were incubated at room temperature for 1 h. The bacteria were washed twice with PBS and filtered with Whatman BLACK NUCLEPORE membranes, pore size 0.2 μm. The filters were washed twice with PBS, moved to microscope slides and sealed with a drop of immersion oil. The slides were kept dark.

Basic ELISA protocol. A basic protocol is described below, but the exact conditions in each experiment are described in context of the results described below. 0 to 10⁷ bacteria suspended in buffer were introduced in the mucus coated wells. Unless otherwise stated, 0.1 mg/ml mucus concentration and 10⁷ bacteria/well were used. In some experiments, Bio-Mos (Alltech, Nicholasville, Ky.) was added together with the bacteria diluted in PBS at the below described concentrations.

The plates were incubated at +37° C. or room temperature for 1 hour. The plates were washed with buffer (PBS or HEPES-Hanks, 300 ul), three times. Unspecific binding was blocked either with milk, fetal bovine serum or BSA (bovine serum albumin) in PBS. The plates were incubated for 1 hour at +37° C. or room temperature. The blocking buffer was removed and primary antibody was introduced in dilutions between 1:200 and 1:100 000. The antibody was diluted in blocking buffer. The plates were again incubated for 1 hour at +37° C. or room temperature and washed three times with PBS or HEPES-Hanks Secondary antibody was added in dilutions between 1:1000 and 1:100 000 in blocking buffer. The plates were incubated for 1 hour at +37° C. or room temperature. After the last incubation, the plates were washed five times with PBS (300 ul/well) to ensure that all free secondary antibody was completely removed. Substrate was added and the plate read with an ELISA reader and/or photographed.

### Example 2

### Intestinal Mucus Concentration

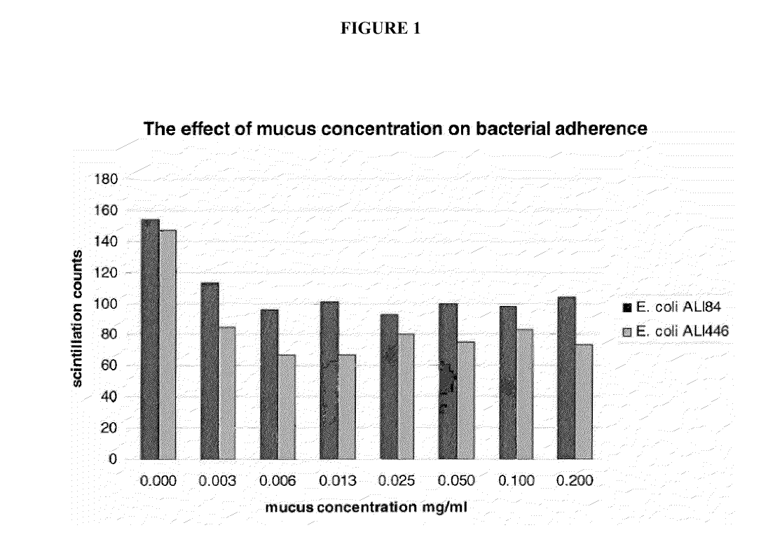

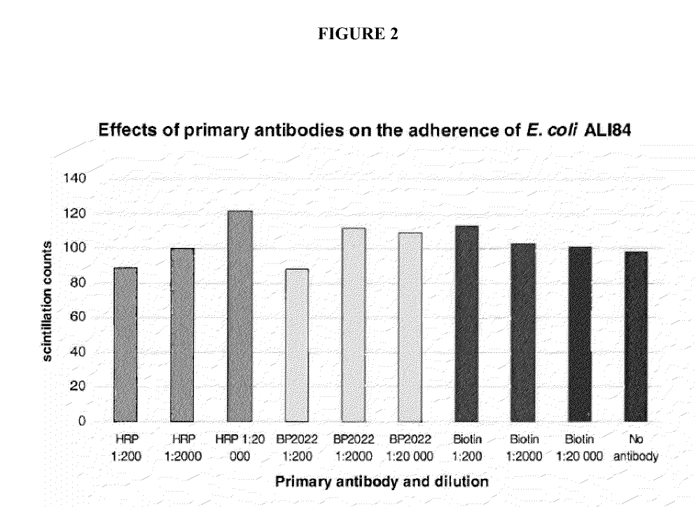

In order to identify concentrations of mucus in the coating suspension, wells were coated with suspensions with different mucus concentrations. Mucous concentration was a variable identified to be important for assay reliability (e.g., if the mucus concentration is too low the bacteria may bind to the plate instead of mucus). Basic method: Radioactive attachment assay (see above Materials and methods). Plates: 96-well PET plates (soft plates); Mucus: Pig proximal ileum, 0.0-0.2 mg protein/ml coating buffer; Bacteria: E. coli strain ALI84 on one plate, E. coli strain ALI446 on the other plate; 10⁷ bacteria/well. Buffer: HEPES-Hanks; Blocking: No blocking; Primary antibody: None; Secondary antibody: None; Incubation temperature: +37° C. The effect of mucus concentration of E. coli ALI 84 and AL1446, as measured by radioactively-labeled bacteria in shown in FIG. 1.

FIG. 1 indicates that the bacteria showed optimal adherence to the wells with no mucus. In order to verify that the bacteria attach to the mucus and not to the plate, a relatively high mucus concentration (0.1 mg protein/ml coating buffer) was identified and chosen for subsequent experiments. Thus, in some embodiments, the present invention utilizes a suitable mucous concentration for coating wells (e.g., an amount that reduces and/or eliminates bacteria binding to plate wells).

### Example 3

### Analysis of Inhibitory Effects of Primary Antibodies on Bacterial Adherence

To test if the primary antibodies influence the adherence of bacteria, bacteria in mucus coated wells were incubated with antibody dilutions ranging from no antibody to 1:200. Basic method: Radioactive attachment assay (see Materials and methods). Plates: 96-well PET plates (soft plates); Mucus: Pig proximal ileum; Bacteria: E. coli strain ALI84 on one plate, E. coli strain ALI446 on the other plate. 10⁷ bacteria/well; Buffer: HEPES-Hanks; Blocking: No blocking; Primary antibodies: HRP=HRP-conjugated anti-E. coli; BP2022=unconjugated anti-E. coli, Biotin=biotin-conjugated anti-E. Coli; Secondary antibodies: No 2nd antibody; Incubation temperature: +37° C.

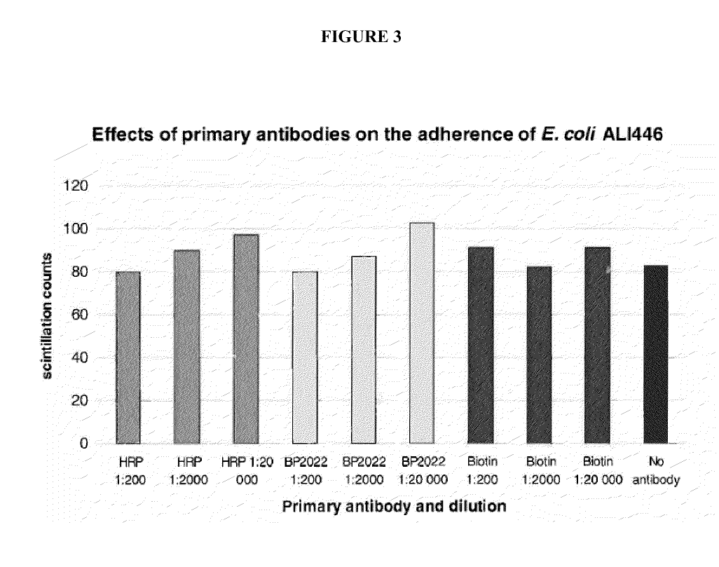

As described in FIGS. 2 and 3, two different first primary antibodies enhanced the adherence of the bacteria to mucus at the lowest concentration (diluted 1:20000) but slightly reduced the adherence at the 1:200 dilution. It is possible that the Fc region of the two first antibodies is free to attach to the mucus, whereas the binding of this region to the mucus is blocked by biotin in the third antibody. At a low concentration, the two first antibodies may act to associate the bacteria and mucus (Fab region binding to bacteria and Fc region binding to mucus). At higher concentrations, (dilution 1:200), the binding of bacteria to the mucus is reduced as antibodies are apparently blocking mucus binding sites on the bacterial surface (or bacteria binding sites on the mucus). The biotin-conjugated antibody had a very small effect; it appears to have enhanced adhesion. Furthermore, in this experiment, primary antibody was added together with the bacteria, but in the colorimetric attachment ELISA methods described herein, the bacteria are first incubated alone for 1 hour. Thus, in some embodiments, the effect of antibodies on bacterial adherence/binding (e.g., antibody actually increasing or decreasing bacterial adherence) is reduced and/or eliminated when the bacteria have attached to the mucus (e.g., in the absence of antibody). Also, in some embodiments, fetal bovine serum or other blocking agent can be utilized for blocking unspecific binding. Thus, in some embodiments, the present invention provides that HRP-conjugated primary antibody and non-conjugated primary antibody alter (e.g., enhance or inhibit) adherence of bacteria to mucus in a conventional, radioactive attachment assay, and that the colorimetric assay of the present invention does not suffer from such alteration. Furthermore, as all antibodies tested appeared suitable for methods described herein, the present invention also provides that binding/attachment assays of the invention are suitable with a wide variety of antibodies (e.g., non-conjugated and conjugated antibodies).

### Example 4

### Non-Specific Color Development by E. coli with ELISA-Substrates

The ability of the two selected strains of E. coli to produce an non-specific color reaction with ELISA-substrates was tested. The experiment was carried out in microfuge tubes incubating bacteria with the substrates. A microfuge method (See Example 1 Materials and methods) was utilized.

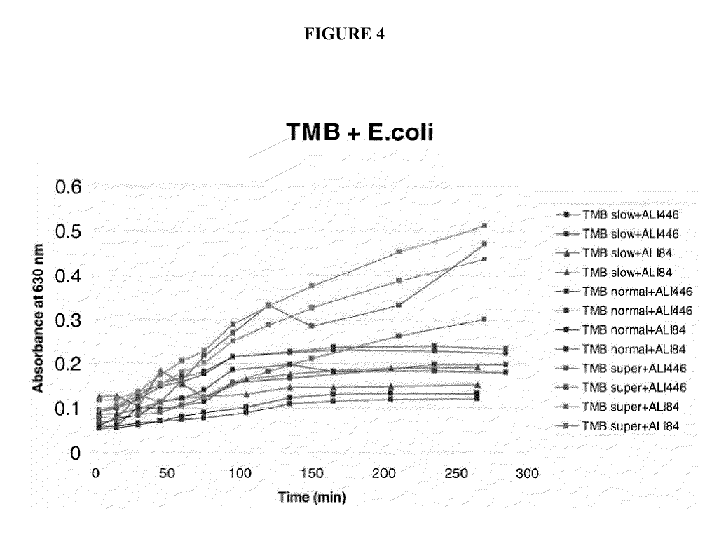

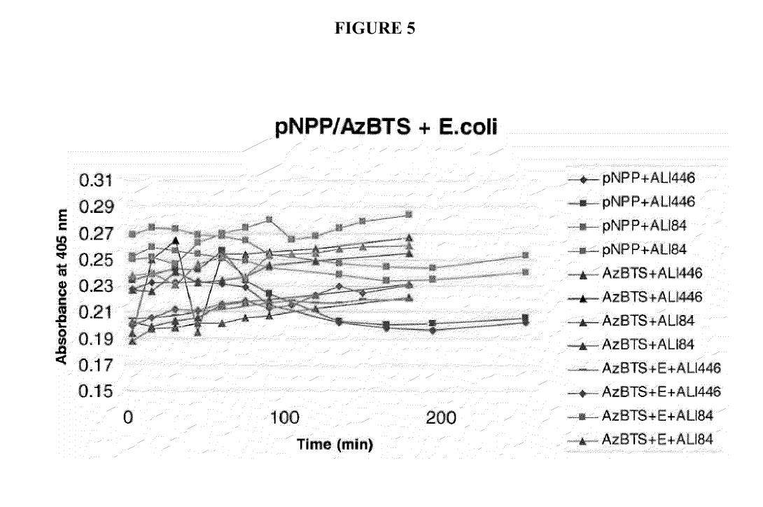

As shown in FIGS. 4 and 5, the color produced by the bacteria was minimal and comparable to the color development of the substrate only. Within a time frame of about 5 to 30 minutes, none of the substrates produced a significant color reaction. Thus, in some embodiments, the present invention provides that each of the ELISA substrates described herein is suitable for use in an adherence/attachment assay of the invention.

### Example 5

### Non-Specific Color Development by Mucus with ELISA-Substrates

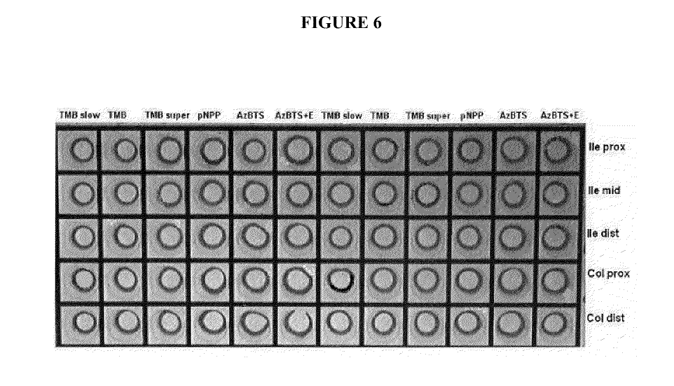

Five mucus samples from a −I year old pig were tested for their ability to produce an non-specific color reaction with the ELISA substrates. An ELISA was performed as described in Example 1 Materials and methods. Plate: Soft plate; Mucus: Pig proximal ileum, mid and distal ileum and proximal and distal colon; Bacteria: No bacteria; Antibodies: No antibodies; Buffer: PBS; Blocking: 1% BSA1PBS 1 hour; Incubation temperature: +37° C.; Substrate: All six substrates shown in FIG. 6.

As shown in FIG. 6, the mucus from pig ileum but not colon produced a color reaction with para-nitrophenyl phosphate (pNPP). Other substrates did not react with the mucus. The color production of pNPP may be due to an intrinsic phosphatase present in the ileal mucus. Thus, the present invention provides, in some embodiments, mucus from pig ileum but not colon produces a color reaction with para-nitrophenyl phosphate (pNPP). Other substrates did not react with the mucus.

### Example 6

### Primary Antibody Specificity Testing

The specificity of the primary antibody was tested with fluorescence microscopy. In brief, the bacteria were blocked with BSA, incubated first with a primary and then with a secondary, FITC-conjugated antibody. The fluorescence was visualized with a fluorescence microscope. The FITC method described in Materials and Methods of Example 1 was utilized.

All samples with the secondary antibody showed some fluorescence even if primary antibody was absent, and the concentration of the secondary antibody produced the most remarkable differences between the samples. Thus the present invention provides that, in some embodiments, non-specific binding can be attenuated and/or inhibited using fetal bovine serum (FBS) and/or bovine serum albumin (BSA)

### Example 7

### Non-Specific Binding of Antibodies to Mucus or Plate

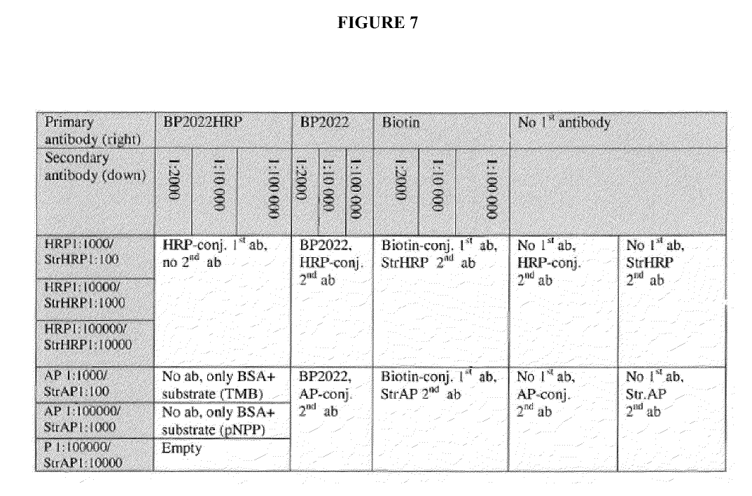

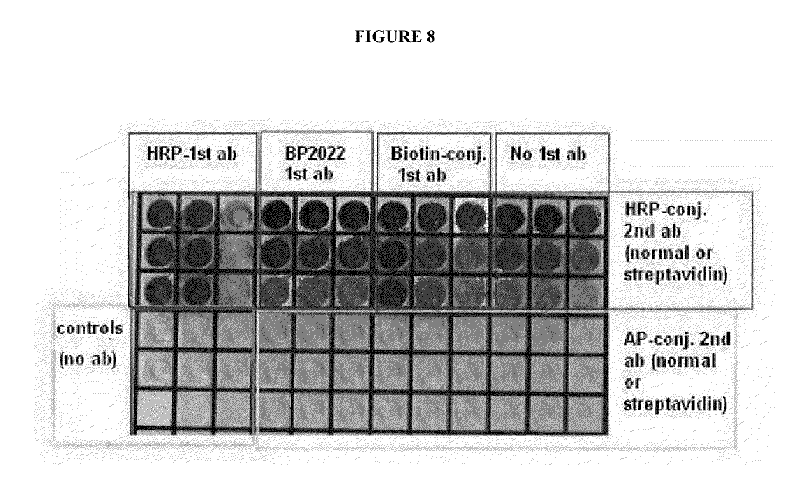

To test if antibodies bind non-specifically to the plate or mucus without bacteria, primary and secondary antibodies were added into a mucus coated plate. The ELISA method described in Example 1 was utilized. BSA was used for blocking non-specific binding. Plate: MaxiSorp plate; Mucus: Pig proximal ileum; Bacteria: No bacteria; Blocking: 1% BSA in PBS; Buffer: PBS; Primary antibodies: see Table 1; 200 ul of different dilutions in 1% BSA in PBS; Secondary antibodies: see Table 1; 200 ul of different dilutions in 1% BSA in PBS; Incubation temperature: +37° C.; Substrates: TMB (for HRP-conjugated 2nd antibodies), pNPP (for AP-conjugated 2nd antibodies). FIGS. 7 and 8 shows the plate layout for testing of non-specific binding of antibodies to mucus or plate, and results of non-specific binding, respectively. No bacteria were used in the experiment. Thus, in some embodiments, the present invention provides a strong non-specific binding of the antibodies to the mucus or plate. In some embodiments, BSA is not an appropriate blocking agent for the attachment assays of the invention.

### Example 8

### Testing Blocking Agents to Prevent Non-Specific Binding of Antibodies

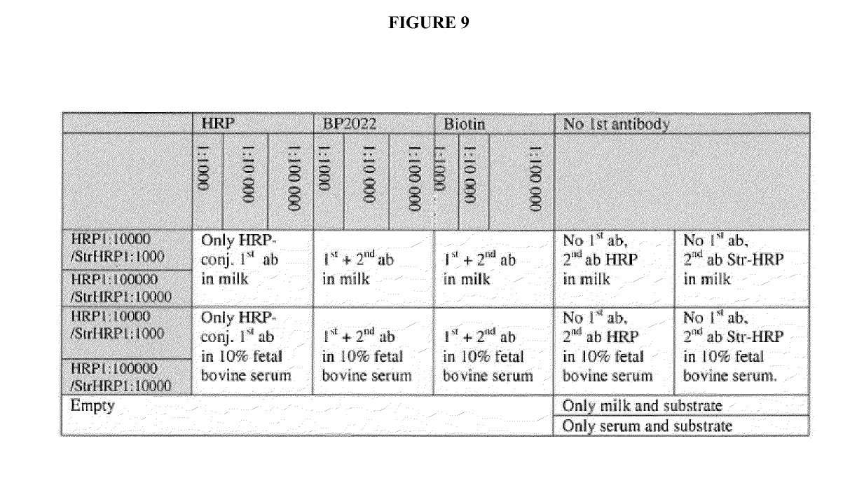

The secondary antibody bound strongly to mucus/plate without addition of any bacteria or primary antibody (See FIG. 8). Thus, milk and fetal bovine serum were tested in place of BSA for blocking the non-specific binding. The basic ELISA method described in Example 1 was utilized. Plate: MaxiSorp plate; Mucus: Pig proximal ileum; Blocking: 5% non-fat milk powder in PBS or 10% fetal bovine serum in PBS for 1 h at +37° C.; Buffer: PBS; Primary antibodies: see Table 2; 200 ul of different dilutions in the blocking buffer; Secondary antibodies: see Table 2; 200 ul of different dilutions in the blocking buffer; Streptavidin-conjugated secondary antibody was used in dilutions of 1:1000 and 1:10000, according to the manufacturer's recommendation. Incubation temperature: +37° C.; Substrate: TMB. FIG. 9 shows the plate layout for the assay. Primary antibodies: HRP=HRP-conjugated 1st ab, BP2022=non-conjugated polyclonal anti-E. coli 1st antibody; Biotin=biotin-conjugated anti-E. coli 1st antibody. Secondary antibodies: HRP=HRP-conjugated IgG; StrHRP=HRP-labeled streptavidin. No bacteria were used in this experiment.

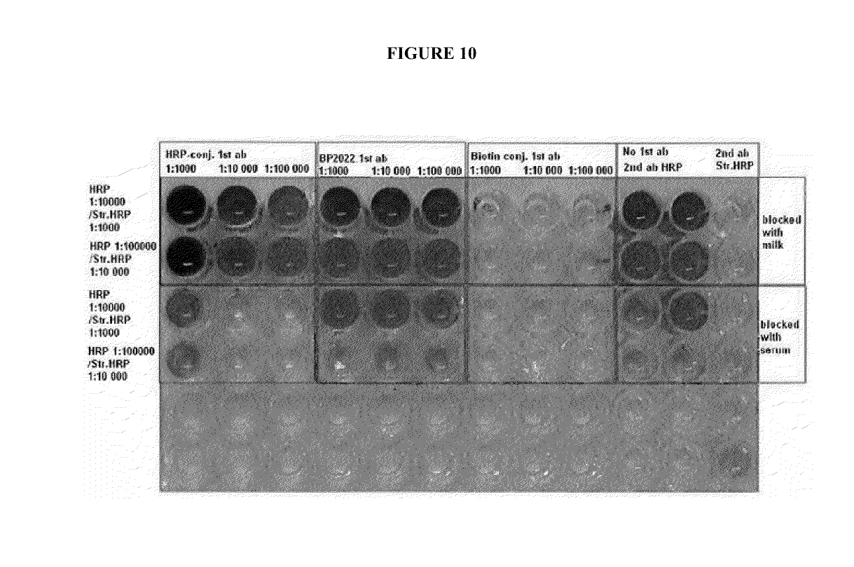

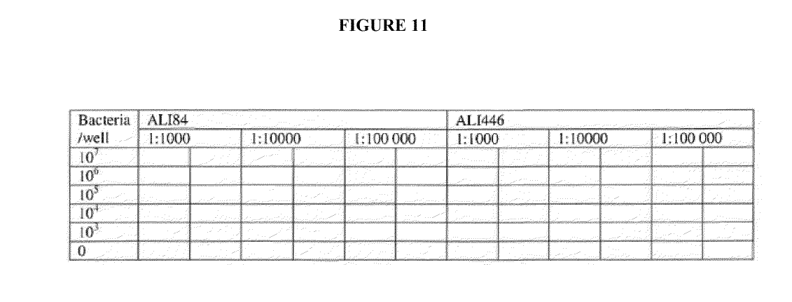

As shown in FIG. 10, fetal bovine serum blocked non-specific binding more efficiently than milk. Non-specific binding was minimal with the biotin-streptavidin-complex and strongest with BP2022-primary antibody. Based on this experiment, 10% fetal bovine serum in PBS was chosen for blocking in experiments. Thus, the present invention provides that 10% fetal bovine serum in PBS was a suitable blocking agent for attachment assays of the present invention.

### Example 9

### Bacterial Binding to Mucus Coated Plates

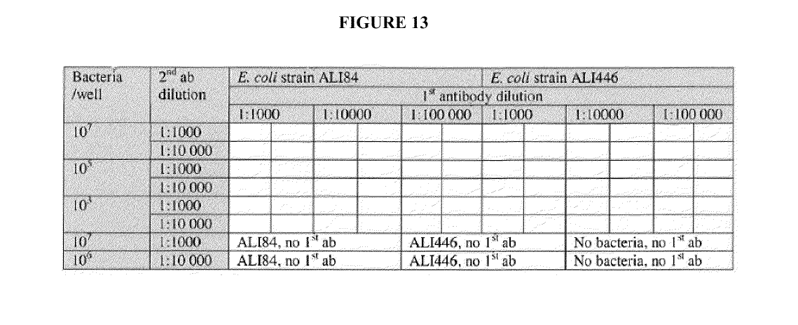

Optimization of the whole ELISA protocol (including both mucus and bacteria) was commenced by optimizing the dilution of the antibodies and the number of bacteria/well. The basic ELISA method described in Example 1 was utilized. Plate: MaxiSorp plate; Mucus: Pig proximal ileum; Bacteria: E. coli strains ALI84 and ALI446, the number of bacteria/well is indicated in FIG. 11. Buffer: HEPES-HanksIPBS; Blocking: 10% fetal bovine serum in PBS; Primary antibodies: 200 ul of different dilutions in the blocking buffer (FIGS. 11 and 13); Secondary antibody: 200 ul of different dilutions in the blocking buffer (FIG. 13); Incubation temperature: +37° C.; Substrate: TMB.

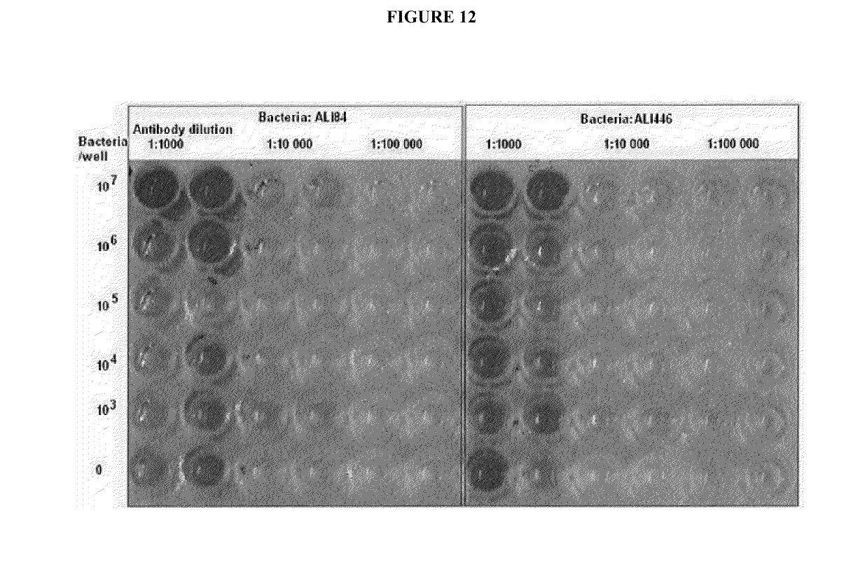

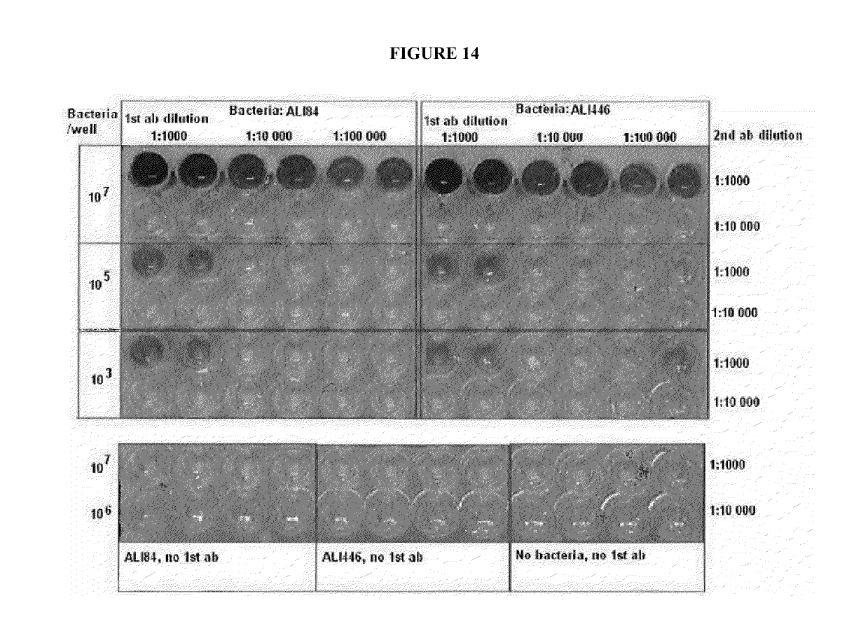

As shown in FIGS. 12 and 14, the biotin-streptavidin complex was more specific than the HRP-conjugated primary antibody. No unspecific binding was observed (See FIG. 14, controls, two separate rows). This confirmed the result obtained in Example 8.

Thus, it was determined to continue with the biotin-streptavidin combination only. FIG. 14 shows that a dilution of 1:1000 was able to produce a reaction on even smaller number of bacteria, and therefore this dilution was chosen for additional experiments. The dilution of the primary antibody was also optimized. FIG. 14 shows that 1:10 000 was too small of a dilution of secondary antibody (streptavidin-HRP) to be useful for detecting a small number of bacteria (thus, in some embodiments, a 1:1000 dilution was utilized).

Biotin-streptavidin combination was more specific than other antibodies and was utilized in subsequent experiments. Thus, the present invention provides that, in some embodiments, 10⁷ bacteria/well is an optimal number of bacteria to utilize in a attachment assay of the invention, although greater (e.g., more than 10⁷ bacteria/well (e.g., 10⁸ bacteria, 10⁹ bacteria, 10¹⁰ bacteria or more)) or fewer (e.g., less than 10⁷ bacteria/well (e.g., 10⁶ bacteria, 10⁵ bacteria, 10⁴ bacteria or less)) can also be utilized. A 1:1000-1:10 000 dilution of primary antibody was optimal in this experiment, but the amount of primary antibody can be above or below this range. In some embodiments, an amount of primary antibody is chosen so as not to limit the color reaction.

### Example 10

### Linearity of the ELISA in Detecting the Number of Bacteria

In order to estimate the linear range of the ELISA method, the ELISA was performed with different numbers of bacteria/well. The basic ELISA method described in Example 1 was utilized. Plate: MaxiSorp plate; Mucus: Pig proximal ileum; Bacteria: E. coli strain ALI84, 107 bacteria/well; Buffer: PBS; Bio-Mos: None; Blocking: 10% fetal bovine serum; Primary antibody: biotin-conjugated primary antibody, 1:1000 dilution in blocking; Buffer; Secondary antibody: HRP-conjugated streptavidin, I:1000 dilution in blocking buffer; Incubation temperature: +37° C. for one plate, room temperature for the other plate; Reagents at room temperature; Substrate: TMB.

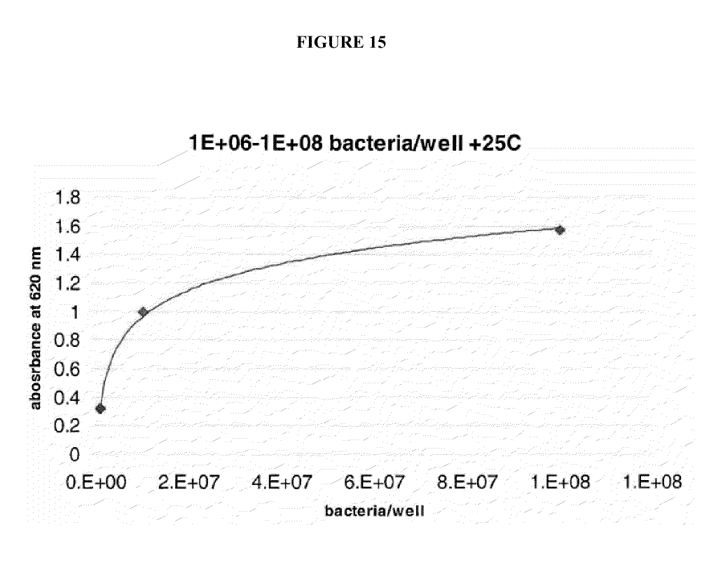

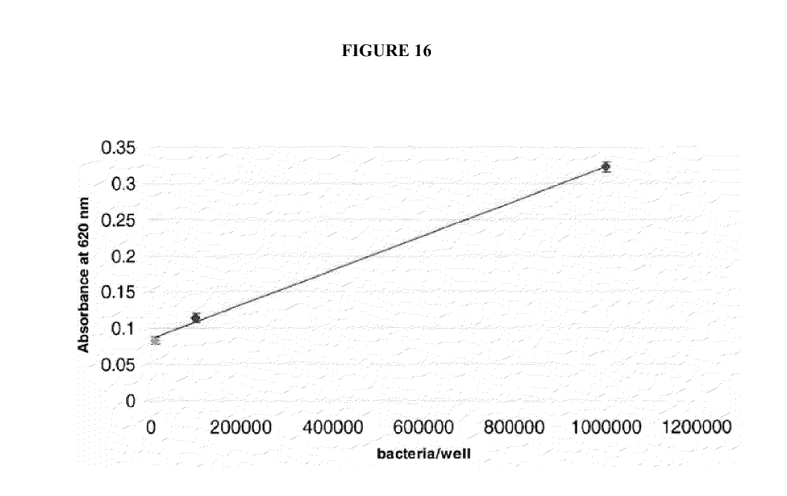

The absorbance at 620 nm versus the number of bacteria was plotted. For bacterial counts up to 10⁶ bacteria/well, the relationship was linear (See FIG. 16), but from 10⁶ to 10⁸, the closest fit trend line was logarithmic (See FIG. 15). Thus, the present invention provides that at a certain number of bacteria/well, the ability of mucus to bind to bacteria decreases as the binding sites become saturated.

Thus, in some embodiments, an assay of the invention is linear through a range of bacterial cell numbers/well (e.g., from 0 through about 10⁶ bacteria per well). In some embodiments, the present invention provides a highly sensitive calculation of bacteria attached per well. In some embodiments, the methods of the invention are standardized (e.g., to achieve repeatable, comparative results).

### Example 11

### The Effect of Bio-Mos on Bacterial Adherence

To test the linearity and resolution of the ELISA-method, different concentrations of Bio-Mos were used to block the adhesion of bacterial cells to mucus. The results using the basic colorimetric ELISA described in Example 1 were compared with results obtained utilizing the radioactive attachment assay also described in Example 1. Plate: MaxiSorp plate and soft plate; Mucus: Pig proximal ileum; Bacteria: E. coli strains ALI84 and ALI446, ˜10⁷ bacteria/well. The same suspension of radioactively labeled bacteria was used for both plates to ensure that the plates were identical; Buffer: HEPES-Hanks, PBS; Bio-Mos: Concentrations 0-20 g/l, diluted in PBS; Blocking (only MaxiSorp plate): 10% fetal bovine serum in PBS; Primary antibody (only MaxiSorp plate): biotin-conjugated primary antibody (1:1000 in blocking buffer); Secondary antibody (only MaxiSorp plate): HRP-conjugated streptavidin (1:1000 in blocking buffer); Incubation temperature: +37° C.; Substrate: TMB.

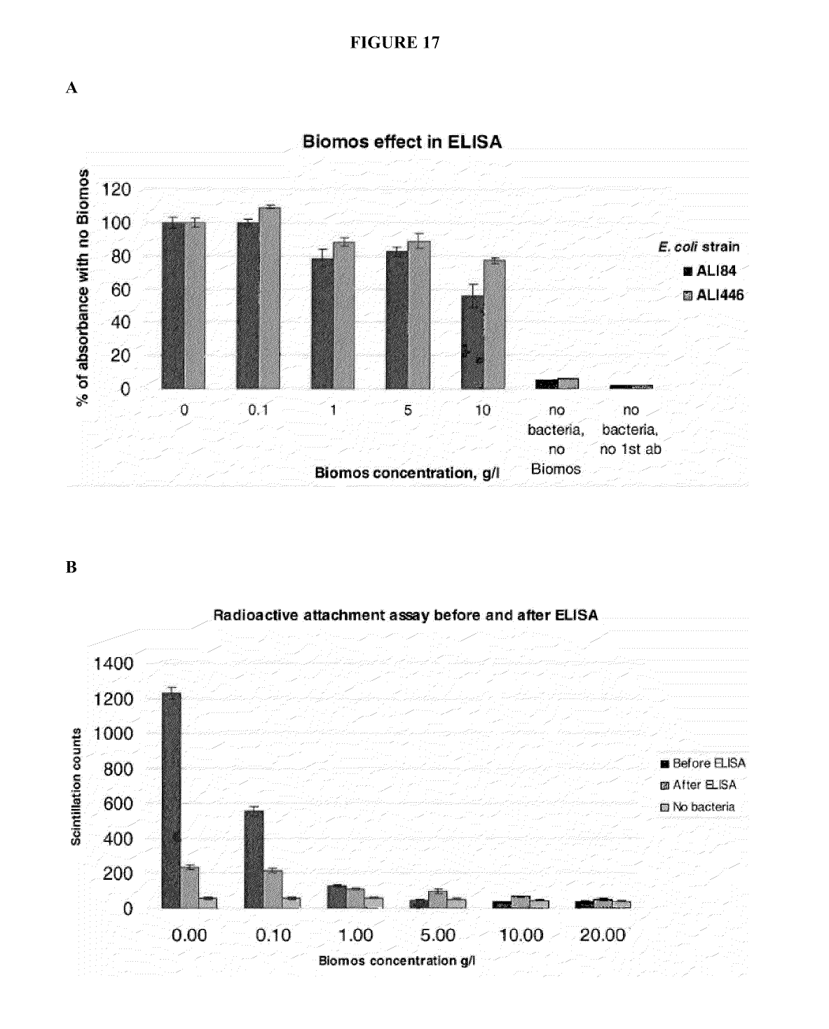

As shown in FIGS. 17A and 17B, the radioactive assay detected differences in bacterial adherence when different concentrations of Bio-Mos were used. After performing the ELISA, both plates were measured with scintillation counter (5 min/well). FIG. 17B shows that the scintillation counts are lower after performing the ELISA, suggesting that bacteria were washed away during the ELISA. However, a clear effect of Bio-Mos was observed.

An unexpected feature of the colorimetric ELISA method was identified in that it was able to detect attachment differences between wells with no bacteria (controls) and wells with the greatest concentration of Bio-Mos (with the smallest number of bacteria) (See FIG. 17A), whereas the scintillation counts of the wells with the greatest Bio-Mos concentration are close to the no bacteria control (FIG. 15). Thus, the present invention provides, in some embodiments, an ELISA method that can detect a attachment/adherence differences between wells with the greatest concentration of Bio-Mos (with the fewest number of bacterial cells) and with no bacteria (controls) (e.g., the colorimetric assay is much more sensitive than the radioactive assay in this concentration range).

### Example 12

### Bacterial Numbers Important for Detecting Attachment Alteration Effects

The aim of this experiment was to find the number of bacteria/well for detecting the effect of Bio-Mos. Basic method: ELISA (see Materials and methods); Plate: MaxiSorp plate; Mucus: Pig proximal ileum; Bacteria: E. coli strains ALI84 and ALI446, 0-108/well; Bio-Mos: Concentrations 0-20 gll diluted in PBS; Buffer: PBS; Blocking: 10% fetal bovine serum; Primary antibody: Biotin-conjugated primary antibody, 1:1000 dilution in blocking buffer; Secondary antibody: HRP-conjugated streptavidin, 1:1000 dilution in blocking buffer; Incubation temperature: +37° C.; Substrate: TMB.

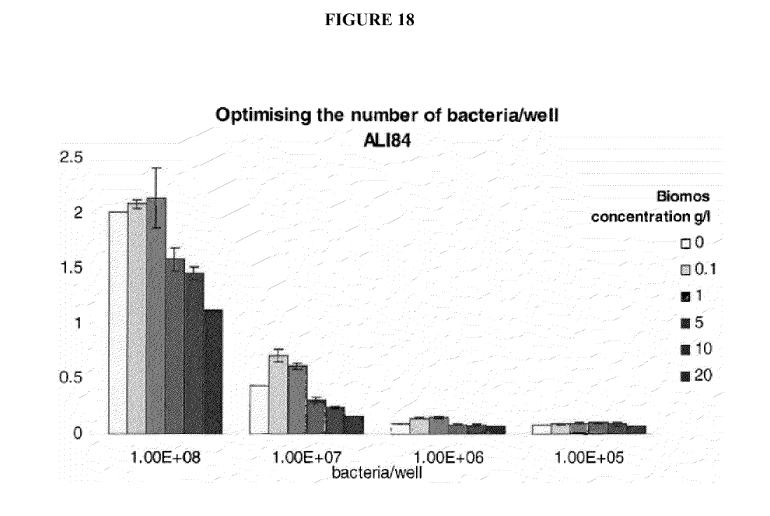

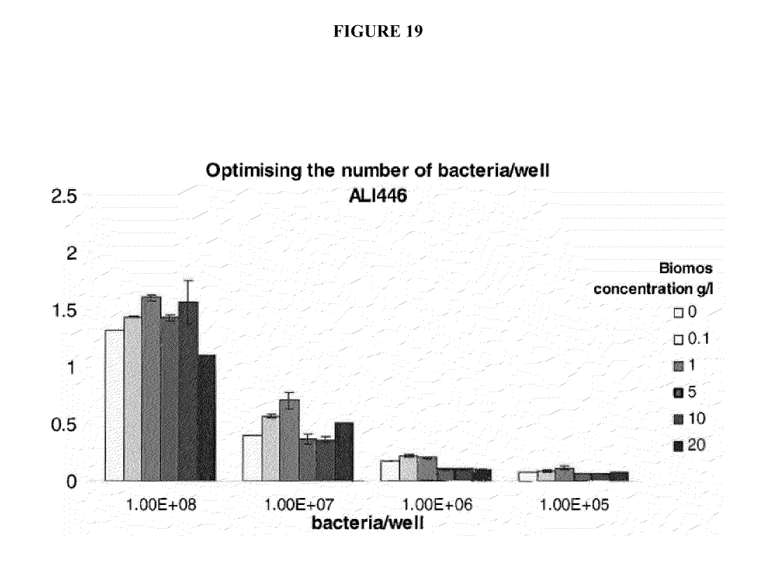

As shown in FIGS. 18-19, it was determined that, in some embodiments, the number of bacteria/well should exceed 10⁵ in order to produce detectable differences. Clear differences can be seen when using 10⁷ bacteria/well. 10⁸ bacteria/well also produces clear differences, but the reaction reaches its endpoint very fast and can thus cause variation as the substrate cannot be added simultaneously to all wells. It is also possible that at very high number of bacteria/well, antibody- or substrate concentrations or mucus binding sites become limiting. Therefore, subsequent experiments were performed with ˜10⁷ bacteria/well. With both E. coli strains, very low concentrations of Bio-Mos appeared to enhance the attachment of the bacteria to the mucus. Thus, in some embodiments, assays of the present invention have identified that relatively low levels (e.g., about 0.1 to about 1.0 g/l) of an inhibiting agent (e.g., Bio-Mos) removes bacteria more efficiently than a larger amount of agent.

### Example 13

### Improved Washing Method

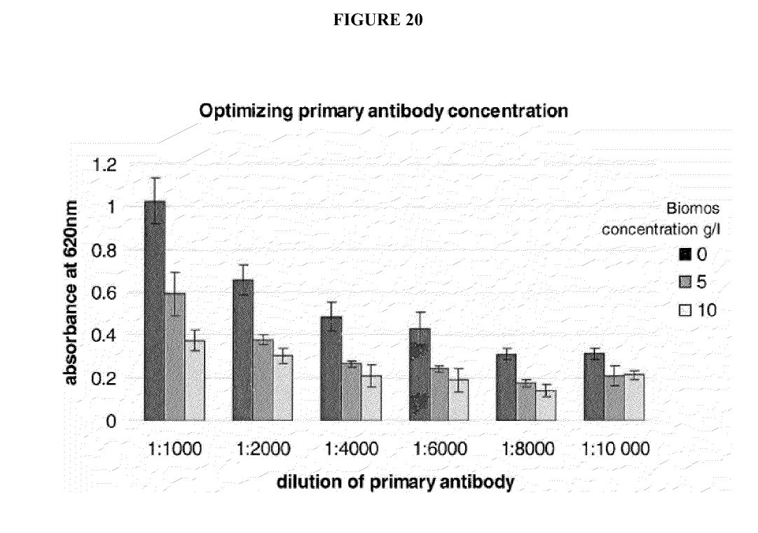

Until now, a Nunc immunowash device was utilized for washing steps, but due to considerable variation between replicate wells, it was determined that this may be too rough of a method. Therefore, a different washing method was tested in order to minimize the variation between samples. The new method was based on shaking away the liquids. Basic method: ELISA (see Materials and methods); Plate: MaxiSorp plate; Mucus: Broiler duodenum; Bacteria: E. coli strain ALI84, 107 bacteria/well; Buffer: PBS; Bio-Mos: Concentrations 0, 0.1, 1 and 10, added together with bacteria; Blocking: 10% fetal bovine serum; Primary antibody: Biotin-conjugated primary antibody, 1:1000 in blocking buffer; Secondary antibody: HRP-conjugated streptavidin, 1:1000 in blocking buffer; Incubation temperature: +37° C.; Substrate: TMB. As shown in FIG. 20, the new washing method (“shake wash”) provided superior results for the ELISA method. The overall signals were greater, and greater differences were detected between different Bio-Mos concentrations. The new method is also faster and allows handling several plates simultaneously. However, it is important that the method be performed under conditions to avoid mixing the contents of the wells. Thus, the present invention provides a new shake-wash method that is superior to conventional washing methods. In the shake-wash method wash buffer is added with a pipette to the well before gently shaking the plate upside down to empty the wells without allowing the solution to transfer from one well to another. This is repeated as many times as necessary.

### Example 14

### Primary Antibody Concentration

Until now, a 1:1000 dilution of the primary antibody had conventionally been utilized in radioactive assays in order to ensure that the antibody concentration did not limit the sensitivity of the ELISA. Thus, if an assay of the present invention is to be utilized in large scale settings, antibody dilution may play a critical role in deployment of the assay. Dilution of primary antibody was tested to determine feasibility of deployment of the assay on a large scale. The basic ELISA describe in Example 1 was utilized; Plate: MaxiSorp plate; Mucus: Pig proximal ileum; Bio-Mos: Concentrations 0, 5 and 10 g/l, diluted in PBS; Bacteria: E. coli strain ALI84, ˜10⁷ bacteria/well; Blocking: 10% fetal bovine serum in PBS; Primary antibody: 200 μl of biotin-conjugated primary antibody (in a range of 1:1000-1:10 000) in blocking buffer); Secondary antibody: 200 μl of HRP-conjugated streptavidin (1:1000 in blocking buffer); Incubation temperature: room temperature; Substrate: TMB.

The 1:1000 dilution provided a good resolution between Bio-Mos levels, although lesser dilutions also worked well. Thus, in some embodiments, a dilution of 1:1000 or less (e.g., 1:900, 1:750, 1:500, or less) is utilized to provide discernable resolution differences in an assay of the invention. Thus, in some embodiments, a dilution of primary antibody is chosen that is capable of saturating all binding sites on the bacteria tested in an assay. In some embodiments, if an assay of the invention is utilized for detecting the effect of very low amounts (e.g., concentrations of about 0.0001 g/l to about 1.0 g/l), of attachment inhibiting agent (e.g., Bio-Mos) even smaller dilution of primary antibody may be utilized (e.g., 1:750, 1:500 or less (e.g., to ensure that antibody concentration does not limit color development). Alternatively, in some embodiments, the number of bacteria per well is reduced to increase color formation.

### Example 15

### Assay Reaction Volume

Colorimetric signal developed extremely rapidly in wells with the greatest number of bacteria. Therefore, it was determined whether a smaller mucus area (e.g., that is capable of binding fewer bacteria (e.g., thereby reducing signal intensity)) would reduce the signal intensity. It was also determined if it is possible to reduce the amount of reagents by reducing the volume needed to fill the coated well. Wells with a smaller mucus area worked well, and the detection limit of bacteria/well was similar to results obtained in earlier examples. The color developed slower than earlier. Thus, the present invention provides that, in some embodiments, the volume of the mucus in a single well can be between about 200 μl to about 300 μl (e.g., depending upon the strength of the signal desired). In some embodiments, the volume of mucus can be less than 200 μl (e.g., 150 μl, 100 μl, or less) or more than 300 μl (e.g., 350 μl, 400 μl, or more). In some embodiments, the present invention provides that smaller volumes of mucus coating permits use of fewer reagents (e.g., about half the amount of reagents is sufficient for wells with 200 μl of mucus compared to the amount required with a well containing 300 μl of mucus) without a loss of assay sensitivity and functionality (e.g., detectable signal). Thus, the present invention provides methods and assays for maintaining assay sensitivity and functionality while concurrently reducing expense related to reagent depletion.

### Example 16

### Mucus Source

In previously conducted experiments (e.g., Examples 1-15), pig proximal ileum mucus had been utilized. In order to determine if colorimetric detection is possible with other sources of mucus, multiple other sources of mucus were tested (e.g., pig proximal ileum, pig distal colon, broiler duodenum and broiler caecum) in the context of the ELISA assay described in Example 1. Plate: MaxiSorp plate; Mucus: Pig proximal ileum and distal colon, broiler duodenum and caecum; Bacteria: E. coli strain ALI84, 10⁷ bacteria/well; Buffer: PBS; Bio-Mos: Concentrations 0, 0.1, 1 and 10, added together with bacteria; Blocking: 10% fetal bovine serum; Primary antibody: Biotin-conjugated primary antibody, 1:1000 dilution in blocking buffer; Secondary antibody: HRP-conjugated streptavidin, 1:1000 dilution in blocking buffer; Incubation temperature: +37° C.; Substrate: TMB.

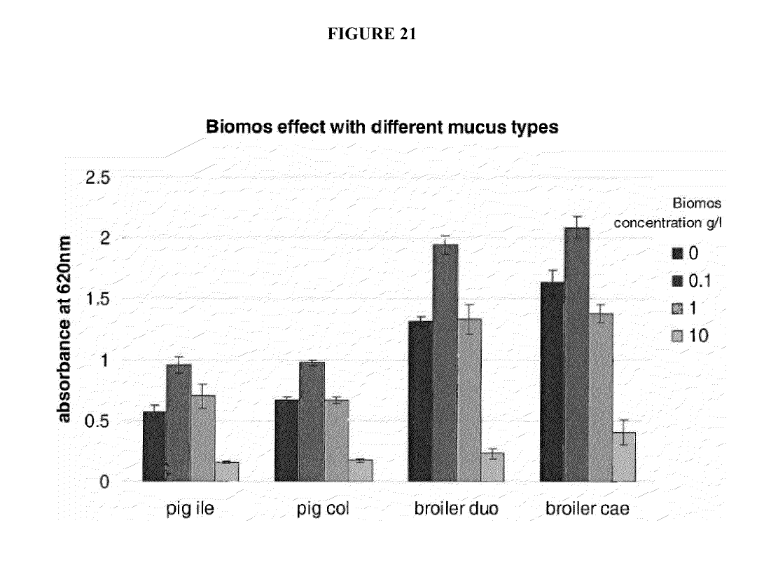

As shown in FIG. 21, bacteria displayed different levels (e.g., strength and/or affinity) of attachment depending upon the type of mucus utilized. For example, bacteria tested displayed almost a two fold greater attachment to mucus obtained from broiler duodenum and broiler caecum than to mucus obtained from pig iliem and pig colon. However, the colorimetric assay was sensitive and robust enough to capture the different levels of bacterial adherence, as well as the ability of a blocking agent (e.g., Bio-Mos) to inhibit bacteria adherence to mucus over a range of blocking agent concentrations. Thus, in some embodiments, the present invention provides a non-radioactive, colorimetric binding assay that find utility with various types and/or sources of mucus (e.g., from different animals and from different portions of the digestive tract (e.g., gut)).

### Example 17

### Radioactive Assay Versus Colorimetric Assay

A side-by-side experiment was conducted to compare materials and methods utilized in the colorimetric assay of the invention with materials and methods utilized in conventional radioactive assays and to assess each assay's ability to detect Bio-Mos induced differences in bacterial adherence. Plates: MaxiSorp plate and soft plate; Mucus: Pig proximal ileum; Bio-Mos: 50 μl/well in different concentrations, diluted in PBS; Bacteria: ˜10⁷ bacteria/well. The same suspension of radioactively labeled bacteria was used for both plates to ensure that the plates are identical; Blocking (only on MaxiSorp plate): 10% fetal bovine serum in PBS; Primary antibody (only on MaxiSorp plate): 100 μl of biotin-conjugated primary antibody (1:1000 in blocking buffer); Secondary antibody (only on MaxiSorp plate): HRP-conjugated streptavidin (1:1000 in blocking buffer); Incubation temperature: Room temperature; Substrate: TMB; After the ELISA, both plates were filled with scintillation liquid and radioactivity was measured with a scintillation counter.

The effect of Bio-Mos was observed to be very similar utilizing the materials and methods of both assays. Low concentrations of Bio-Mos appeared to enhance the attachment of the bacteria to the mucus. Although a mechanism is not necessary to practice the invention and the invention is not limited to any particular mechanism of action, in some embodiments, at the lowest concentrations, Bio-Mos or other type of inhibitory agent participates in the formation of aggregates of the bacteria. Thus, even if the lowest concentration increased the attachment as measured by the assays, it is likely that (e.g., in the context of the lumen of the gut) the aggregates are more easily washed away from the gut. Thus, low Bio-Mos or other inhibitory agent concentration (e.g., identified and/or characterized by methods of the invention described herein) may actually remove bacteria more efficiently than and/or as efficiently as higher concentrations.

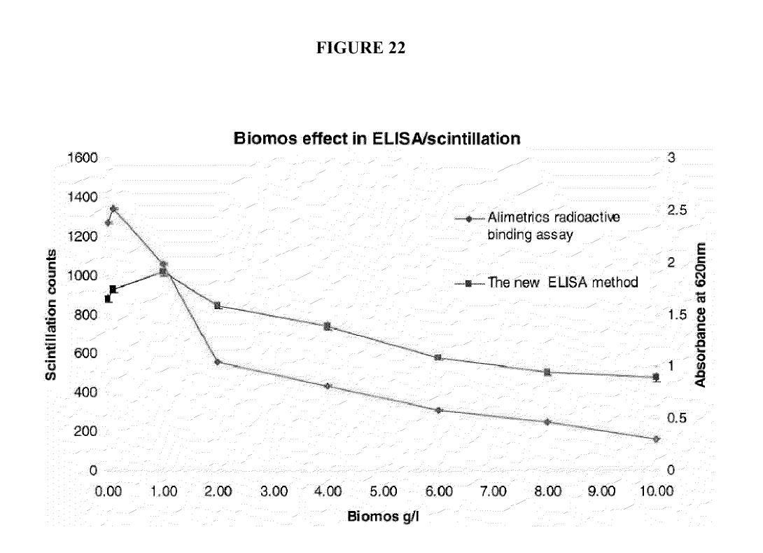

As seen in FIG. 22, at very low concentrations of Bio-Mos, the absorbance of the two methods differed somewhat. The trend of the ELISA line is similar already at very early time points (=low absorbances), and thus the downward trend from 1.0 g/1 to 0.1 g/l is unlikely to result from limitations in the capacity of the ELISA reader. It is possible that color development might be limited by the concentrations of antibodies or substrate, but this hypothesis does not explain the slight downward trend from 1.0 g/l to 0.1 g/l.

These results and those of the other examples show that the highest absorbances are obtained with either 0.1 g/l or 1.0 g/l. Thus, the present invention provides that the variation is caused, in some embodiments, by differences in the number of bacteria between experiments. Thus, in some embodiments, for comparable results, one can use the same bacterial suspension or standardize the culturing method to make sure that the original number of bacteria is similar in parallel experiments. Thus, in some embodiments, reducing the number of bacteria/well might ensure that Bio-Mos and/or other blocking agent (e.g., test agent) concentration is the most limiting factor, instead of the number of mucus binding sites or antibody or substrate concentrations.

### Example 18

### Non-Radioactive Bacterial Binding Assay

Experiments were conducted during the development of embodiments of the invention in order to further test use of the colorimetric ELISA generated herein for measuring pathogen attachment and to assess the degree of test agent alteration of the attachment. Thus, experiments were conducted to determine if the assay could be utilized to screen test agents that may or may not alter (e.g., inhibit) bacterial attachment to mucus. Thus, experiments were conducted to determine if the assay could be utilized to screen test agents that may or may not alter (e.g., inhibit) bacterial attachment to mucus. In short, the present invention provides an alternative to using radioactive, live pathogens (e.g., an assay of the invention need not use live nor radioactive bacteria (e.g., the present invention provides that ethanol-inactivated bacteria can be utilized in an attachment assay)) and also provides air dried, mucus coated plates. Thus, methods developed during development of embodiment s of the invention provide significant benefits over conventional methods in that, in some embodiments, the methods provided herein eliminate the need to use live and/or radioactive bacteria (e.g., pathogenic bacteria) and also eliminate the need to adjust bacterial density each time the assay is conducted.

Storage and uniformity of the bacterial preparation. As described in Examples 9, 10 and 12, the density of bacterial preparation used in the attachment assay was identified as an important variable in the reaction. At low bacterial density the signal was weak, whereas at high bacterial density the signal was above the linear range. From these data, it was determined that for accuracy, sensitivity and comparability of successive assays, a fixed bacterial density was needed. It was also determined whether a standard, bacterial suspension (e.g., for use in a series of assays not taking place at the same time (e.g., on different days, or in different weeks or months)) could be generated (e.g., that was easy to store, use, and that was non-pathogenic). A plurality of bacterial preservation and inactivation methods were identified and tested including preservation and inactivation by chemical fixatives (e.g., ethanol, glutaraldehyde, formalin, DMSO), inactivation by UV irradiation, and use of frozen, live bacterial suspension

Conservation of the mucus-coated plates. In the previously described assays (e.g., Example 1-15), 96-well plates were coated with mucus each time the assay was run. As described, this procedure required overnight incubation. Thus, experiments were conducted to determine if this time consuming process could be replaced (e.g., by pre-coated, long term storable mucus coated plates). A variety of methods were tested including freezing coated plates as well as air drying the plates and using post long term storage (e.g., with different mucus types).

Methods.

Culturing and inactivation of bacteria. Bacteria were grown at 37° C. in Luria broth and transferred in a fresh medium (10% inoculum) one day before intended tests. Multiple different methods of inactivating or preserving bacteria were tested:

Freezing: grown bacterial culture was frozen at −20° C. Before use, the culture was thawed at room temperature. One batch was frozen with glycerol to protect the cells from damage during freezing, but this approach was discontinued as collecting bacteria from glycerol was problematic and produced non-useful results.

Ethanol: 99% ethanol was added 1:1 to bacterial culture in Luria broth to produce a 50% ethanol solution. The suspension was stored at 4° C.

Glutaraldehyde: glutaraldehyde was used at 4% final concentration. The suspension was stored at 4° C.

Formalin: formaldehyde was used at 4% final concentration. The suspension was stored at 4° C.

UV: the culture was irradiated under a UV lamp for 30 or 60 minutes. The suspension was stored at 4° C.

Dimethyl sulfoxide: DMSO was added in the culture at 10% concentration. The suspension was stored at 4° C.

Heat: The culture was heated at 70° C. for 30 minutes and thereafter stored at 4° C.

Bacteria were harvested by centrifugation just before use.

Preparing and conserving the mucus-coated plates. Mucus scraped from the intestine of piglets was diluted in NaCO₃-buffer (pH 9.6) to produce a suspension with 0.1-0.3 mg mucus protein/ml. 300 gl of this suspension was introduced into each well on a 96-well IgA plate. The plate was incubated at 4° C. overnight.

In air-drying, the plates were washed twice with PBS and dried in a laminar flow cabinet overnight. The plates were stored at room temperature in plastic bags. Prior to use, 300 μl PBS was introduced into each well and the mucus was allowed to rehydrate for 10 minutes, after which the PBS was removed by gently shaking the plate upside down.

For freezing, the plates were frozen at −20° C. with the mucus suspension. Prior to use, the plate was thawed at room temperature and washed three times with PBS.

The fresh plates were washed three times with PBS after overnight coating with mucus.

Results

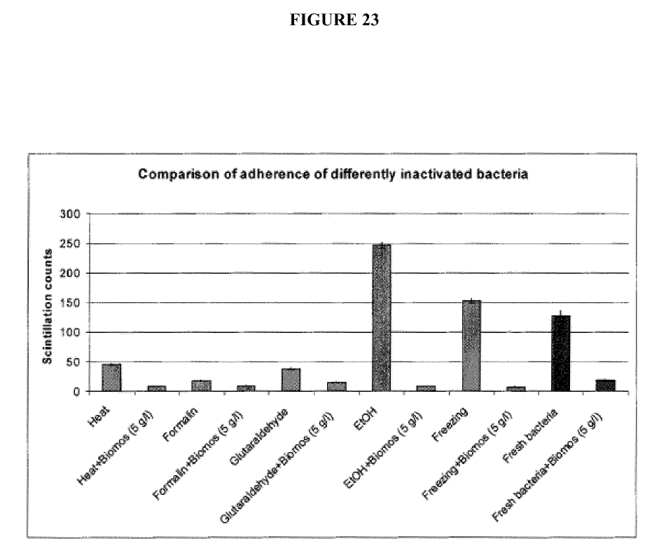

Initial screening of the bacterial conservation methods using the radioactive assay. Three methods using preserved bacteria appeared satisfactory as compared to the assay with fresh bacteria (See FIG. 23). These were ethanol, UV irradiation and freezing (the other methods were identified as being not suitable for the assay). Data for UV and DMSO-inactivated bacteria is not shown in FIG. 23 as different bacterial suspensions were used. DMSO inactivation dramatically inhibited bacterial adherence whereas UV irradiation of the test bacteria yielded relatively good adherence results.

In addition to absolute adherence, the capability of the method to detect the effect of test agent inhibition of attachment was also characterized. The results for the bacterial conservation methods other than DMSO and UV-irradiation are shown in FIG. 23. Regardless of the method chosen, the assay was able to reveal that Bio-Mos inhibited E. coli adherence.

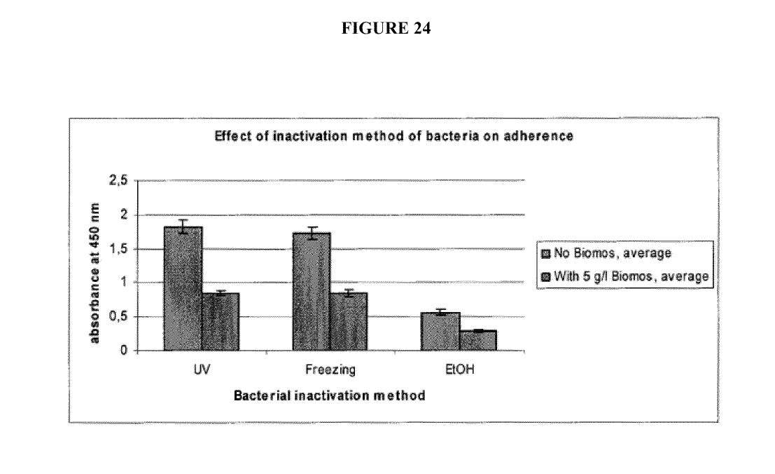

ELISA assays with selected bacterial conservation methods. Based on these results, three bacterial inactivation methods were tested in the ELISA bacterial detection system. While the present invention is not limited to any mechanism of action and an understanding of the mechanism of action is not necessary to practice the invention, it is possible that fixing the bacteria by any of the above-mentioned methods would change antigenic characteristics of the bacteria thus leading to failure of the antibody-based methods to detect the modulated bacteria. However, the ELISA method appeared to work for the tested bacterial preparations. Of the bacterial inactivation methods, the UV-inactivation and freezing were better than ethanol conservation considering absolute bacterial adhesion efficiency, as shown in FIG. 24. However, these methods have major drawbacks when considering practicality of use: UV-inactivated bacterial suspension is stable only when unopened, but is easily spoiled by other bacteria when exposed to ambient microbes. The frozen bacteria, on the other hand, are still alive and may start growing or be metabolically active after thawing. This will influence accuracy and reproducibility of the assay. Furthermore, safety issues have to be considered when working with live bacteria.

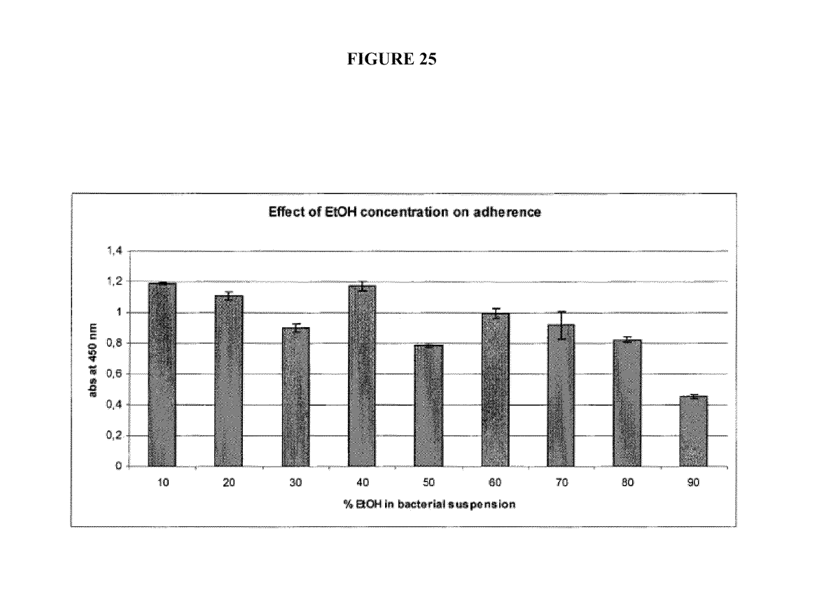

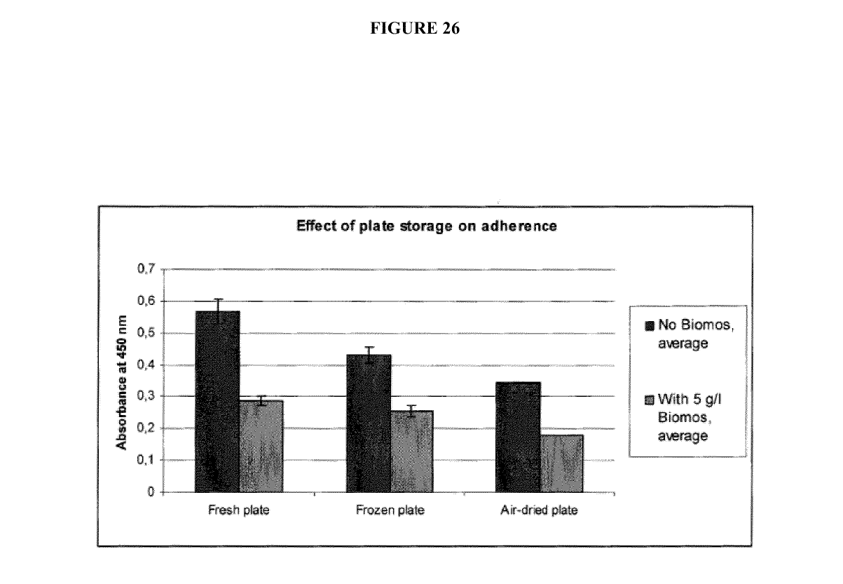

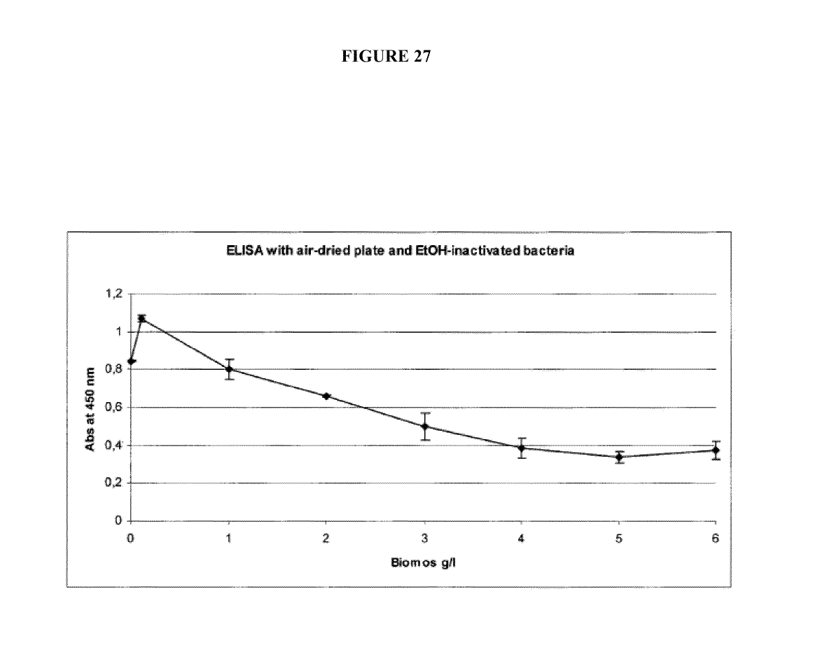

Optimizing the ethanol concentration in bacterial suspension. Even though it appeared that the bacterial conservation by ethanol was not ideal, it was determined to continue to attempt to develop this approach due to the other benefits the method provided. Initially, ethanol at 50% concentration was utilized based on its ability to kill bacteria. However, other alcohol concentrations were tested to determine the effect of alcohol concentration on bacterial adhesion. It was determined that ethanol concentration affected significantly the efficiency of bacterial adherence. While the present invention is not limited to any mechanism of action and an understanding of the mechanism of action is not necessary to practice the invention, in some embodiments, the present invention provides that ethanol detaches or destroys fimbriae essential for the binding, or changes other antigenic properties of the bacteria. Ethanol at a concentration of 40% appeared significantly better than 50% ethanol when considering the efficiency of adherence. The surprising nonlinear trend shown in FIG. 25 was observed repeatedly.

Conservation of the mucus-coated plates. The ability of bacteria to adhere on mucus-coated plates conserved by air-drying and freezing were tested by comparing them with freshly coated plates. In tests with radio-labeled bacteria an air-dried plate was comparable to a freshly coated plate, whereas the frozen plate gave somewhat higher counts. Each method was tested using a non-radioactive ELISA method of the invention.

As shown in FIG. 26, air-dried plates gave a weaker signal than the freeze stored and freshly coated plates. However, the effect of Bio-Mos on bacterial adhesion was clear with all methods used. Thus, the present invention provides a method of using previously generated and stored mucus coated plates (e.g., air-dried or freeze-stored plates). Rehydration time of the dried plates. Time spans from 1 minute to 12 hours were tested to rehydrate the air-dried plates before running the assay. It was determined that rehydration time did not greatly influence the results of the assay, but in order to obtain comparable results, a constant (e.g., 10 minute) rehydration time was utilized for all subsequent assays.

Mucus concentration in the wells. Mucus concentration in the wells had been tested previously, but it was decided to test whether the adherence of the bacteria could be enhanced with a higher mucus concentration. Three levels of mucus in the coating buffer were tested; the levels corresponded to 0.1 mg/ml, 0.2 mg/ml and 0.3 mg/ml of protein, respectively. The adherence of bacteria clearly improved when the mucus concentration was increased, 0.3 mg protein/ml yielding the highest adherence.

Detecting an alteration in bacterial adherence using a test agent (e.g., Bio-Mos). Based on the preliminary scintillation and ELISA experiments, ethanol-inactivated bacteria and air-dried plates were utilized to test the effect of a test agent (e.g., Bio-Mos) and its ability to alter adherence of bacteria to mucus. Data obtained showed that the resulting curve was highly similar to the curve obtained when conducting ELISA with fresh bacteria and plates.

Applicability to other mucus types. The ELISA (using stored plates and EtOH inactivated bacteria) was tested with other mucus types. The ELISA produced useful data using multiple types of mucus including pig proximal ileum, pig distal colon, broiler duodenum and broiler caecum. The present invention provides that ELISA using stored plates and EtOH inactivated and stored bacteria provides useful data regarding, and the effect of a test agent ability to block bacteria attachment/adhesion (e.g., Bio-Mos) was similar regardless of the source of mucus. Thus, the present invention provides compositions (e.g., ethanol-inactivated bacteria and air-dried mucus coated plates) and methods (e.g., non-radioactive ELISA) useful for monitoring and characterizing bacterial cell attachment/adhesion to mucus that is easy to use, safe, and the materials are easily stored and transported.

Further characterization of the non-radioactive bacterial binding assay. Experiments were conducted during development of the invention in order to determine if the non-radioactive assays provided herein were reliable, reproducible and minimized variability. Variation between different batches of bacteria and plates was tested and the reproducibility of the assay was determined (e.g., by running on different days (between plate-variation) and monitoring in plate-variation of replicate samples).

Methods

Culturing and inactivation of bacteria. The bacteria were grown at 37° C. in Luria broth and transferred in a fresh medium (10% inoculum) one day before inactivation with ethanol. Bacteria were inactivated and preserved by adding ethanol directly in the overnight grown culture to the final concentration of 40% vol/vol. The suspension was stored at 4° C. and harvested by centrifugation just prior to use. The pellet was suspended in the original volume of Luria broth to obtain a suspension with approximately 10⁸bacteria/ml.

Preparing and conserving the mucus-coated plates. Mucus scraped from the intestines of piglets was diluted in NaCO₃ buffer (pH 9.6) to produce a suspension with 0.3 mg mucus protein/ml. 300 μl of this suspension was introduced into each well on a 96-well IgA plate. The plate was incubated at 4° C. overnight. The plates were then washed twice with PBS and dried in laminar flow cabinet overnight and stored at room temperature in plastic bags. Prior to use, 300 μl PBS was introduced into each well and the mucus was allowed to rehydrate for 10 minutes, after which the PBS was removed by gently shaking the plate upside down.

ELISA method. Mucus-coated, air-dried plates were allowed to rehydrate for 10 minutes with PBS before adding 100 μl of bacterial suspension and 100 μl of either Bio-Mos suspension in PBS or 100 μl of pure PBS. The samples of treatments were assigned randomly to the wells to avoid systematic errors. The plate was incubated for 1 h at room temperature, protected from light and evaporation. After the incubation, the plate was washed three times with PBS and the blocking buffer (10% fetal bovine serum) was added. The plate was incubated as described above and emptied. Primary antibody was added, the plate incubated, and washed as described above. Secondary antibody was added and the plate was incubated and washed as described above. Finally, TMB-substrate was added to the wells and the color was allowed to develop for 15-30 minutes. The reaction was stopped with sulfuric acid and absorbance measured at 450 nm.

Statistical analyses. Coefficients of variation were estimated for values that were scaled so that the average of zero samples for each plate was 100%. Within plate and between plate estimates for coefficients of variation were then calculated for these scaled values of levels 1 and 2 using the MSEs of one-way ANOVA for between and within treatment as variance estimates. This estimation was performed separately for different levels of Bio-Mos. Power analysis was done to provide an estimate on how many replicates would be needed to detect a difference between two treatments. Risk level u=0.05 and power=0.8 or 0.9 were used.

Results

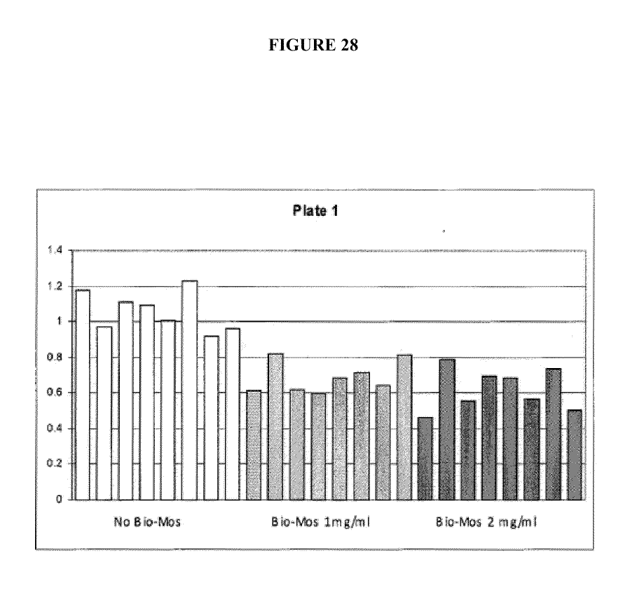

Variation measured from the replicate samples analyzed in the same plate. For the product evaluation purposes it is important that when run in several replicates in the same plate, the assay is repeatable, and thus provides a reliable test, the detection limit of which is known and considered sufficient for the practical use of the assay. For this purpose, the assay was run in the plate coated with homogenous mucus, and with a single batch of the bacterial preparation. FIG. 28 shows that there is some well-to-well variation, but the adherence inhibiting effect of the added Bio-Mos was clear and repeated. Comparison with the control wells showed that the levels 1 mg/ml and 2 mg/ml of Bio-Mos differed from the control with the p-value<0.0001. However, the two Bio-Mos levels did not differ from each other as shown in FIG. 28. Coefficients of variation were calculated separately for each treatment.

Variation Measured from Four Different Mucus Plates

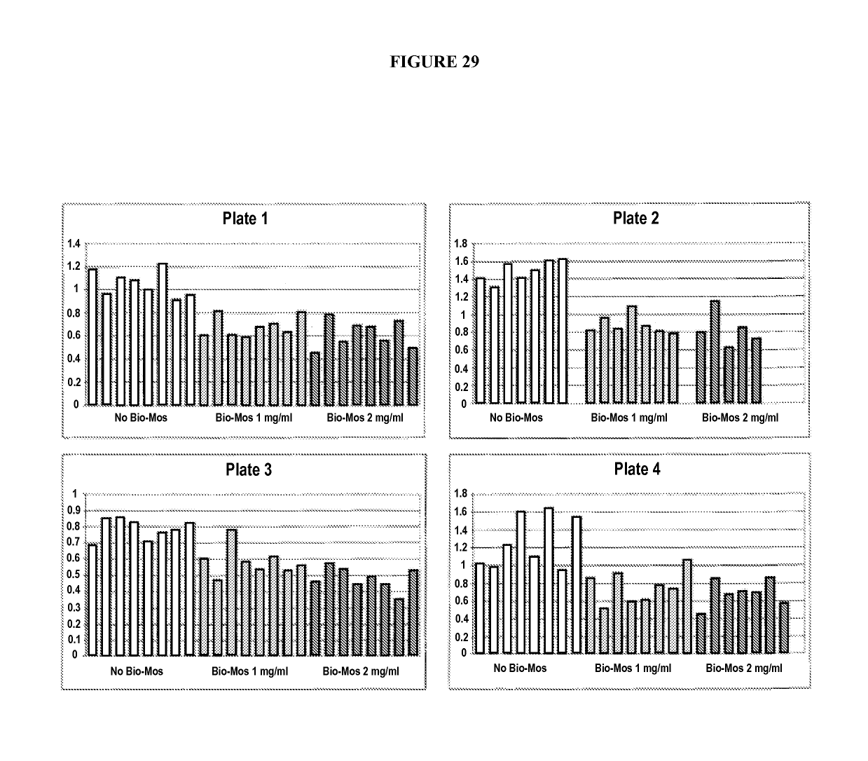

For the product evaluation purposes it is important that the assay is repeatable. In order to determine if the assay was repeatable (e.g., at different times using the same reagents), the experiment in which the data is shown in FIG. 28 was repeated 4 times on four different days. Each set of tests was carried out on a different day and mucus plate, but with a single bacterial preparation. The results are illustrated in the four panels of the FIG. 29. The average variation within different plates were 14%, 14% and 13% for the plates 1, 2 and 3, respectively, and as high as 22% for the plate 4. Table 1, below, shows the CVs calculated for each test within each plate. There appeared to be a trend that the CV was lower for the control wells than for those with Bio-Mos. The average CV calculated from all four test plates and all treatments was 16% (Table 1).

TABLE 1

| | CV measured from the indicated test replicates | | | | |
|---|---|---|---|---|---|
| Test | Plate 1 | Plate 2 | Plate 3 | Plate 4 | Mean |
| No Bio-Mos | 1% | 8% | 8% | 23% | 1% |
| Bio-Mos 1 mg/ml | 13% | 12% | 15% | 24% | 16% |
| Bio-Mos 2 mg/ml | 19% | 23% | 14% | 21% | 19% |
| All | 14% | 14% | 13% | 22% | 16% |

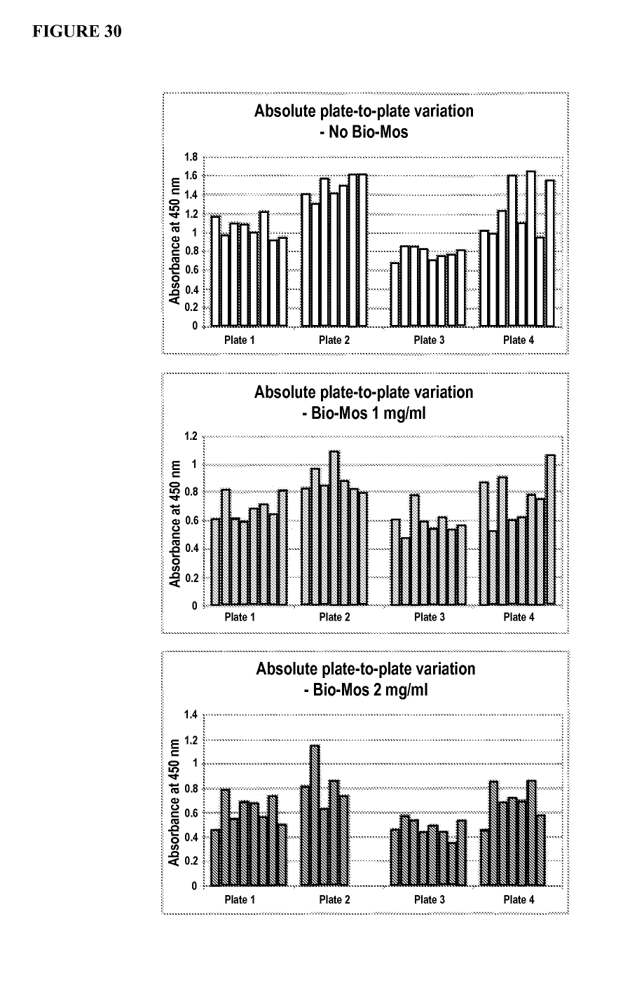

Difference in the absolute signal measured from four different mucus plates. FIG. 30 shows the data in the FIG. 29, but arranged in a different way to emphasize the magnitude of the absolute signal in different tests. The plates were handled similarly but independently. The samples were assigned randomly to the wells to avoid systematic errors due to factors such as well position. It is noteworthy that the absolute levels of signals vary from day-to-day even though the attempt has been to repeat every step of the assay exactly in the same way. While the present invention is not limited to any mechanism of action and an understanding of the mechanism of action is not necessary to practice the invention, in some embodiments, the variation is due to one or more steps including: 1. Mucus binding 2. Washing of the wells 3. Bacterial binding 4. Washing off the free bacteria 5. Binding of the primary antibody 6. Washing 7. Binding of the secondary antibody 8. Color development reaction. However, the present invention provides that although there is some variation in color development, the variation in limited and does not inhibit the generation of useful data (e.g., regarding the ability of one or more test agents to alter (e g , inhibit) bacterial binding to mucus).

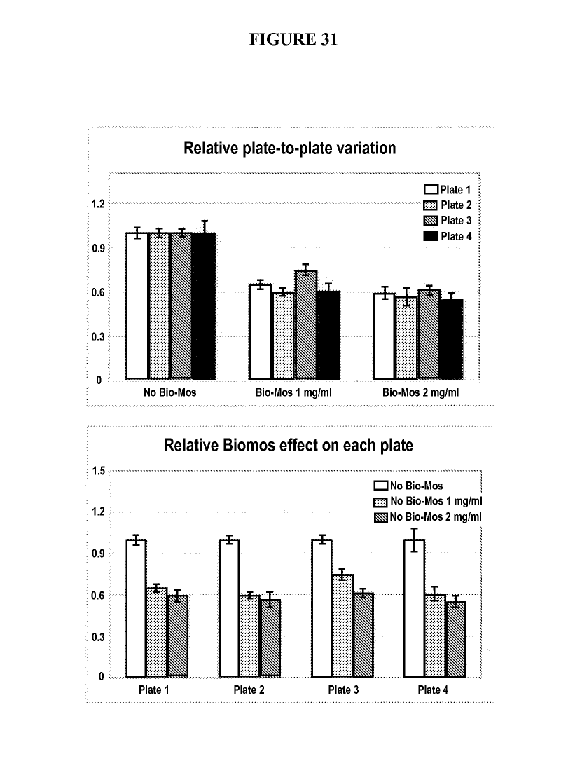

Comparison of plate-to-plate variation when using relative signals. If and when the relevant control treatments were present in the same plate as the products to be tested, the plate-to-plate signal variation is not be problematic. All the signals were changed to relative values by giving the mean of all control wells the value of 1, and the wells with test compounds values normalized to that. The normalized results are shown in FIG. 31. When presented as relative signals it was observed that the detected magnitude of the Bio-Mos effect was nearly identical in all plates.

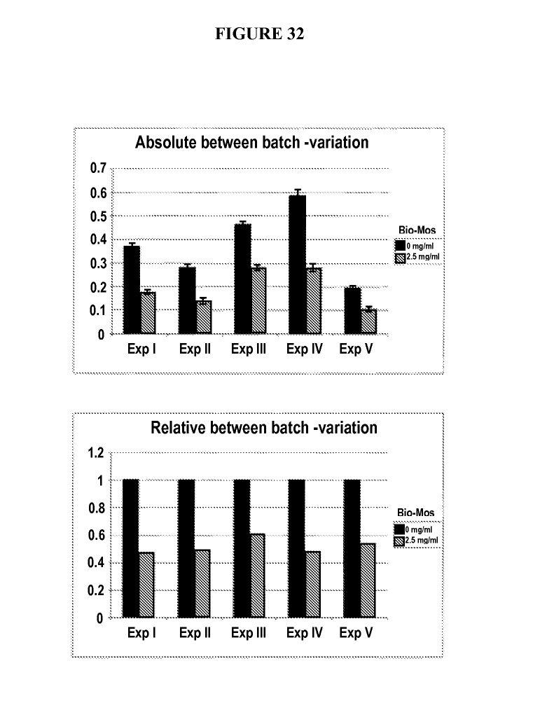

Variation between batches of bacteria. In order to test variation between batches of bacteria and plates, multiple, independently grown bacterial cultures were generated, and multiple, independently made, mucus-coated plates were also generated. The batches were produced completely independently, using different batches of PBS, Luria broth and growing each culture from a new frozen storage bead. A similar ELISA assay was performed with each of the independent plates and cultures. The results of this experiment are shown in FIG. 32.

As shown in FIG. 32, the absolute levels of the signals varied from experiment to experiment, but when compared to the signal of the control tests, the totally independent studies produced data characterizing nearly the identical effect of Bio-Mos on attachment (See FIG. 32)

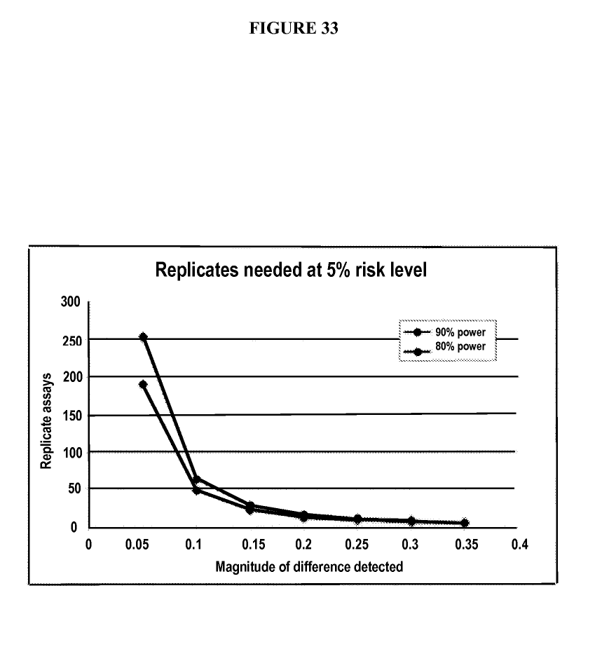

Power analysis. Using the experimental data, it was possible to estimate the numbers of replicates required to achieve a desired detection power. By detection power it is meant the percent difference between the responses of two test compounds or treatments that provides a statistically significantly difference from each other. FIG. 33 and Table 2 below show the relationship between the detection power and the number of replicates.

The present invention provide that if batches of test agents are being compared, as few as 5 (or less) replicates are enough to be able to state that two agents (or dilutions thereof) are different when the product A is, for example, inhibiting adherence by 50%, whereas the product B is inhibiting it by 66%, then 5 replicates would be enough.

TABLE 2
Examples of detection power

| Number of | Difference |
|---|---|
| replicates | detected |
| 5 | 31% |
| 10 | 22% |
| 15 | 18% |

Based on 80% power at 5% risk level

### Example 19

### Generation of Stable Functional Bacterial Preparations and Mucus Coated Plates

Experiments were conducted during development of embodiments of the invention in an effort to prepare a bacterial suspension that possessed the following characteristics: fimbriae present and/or retained on prepared bacteria capable of mediating bacterial adherence on mucus, wherein the fimbriae are numerous and structurally intact permitting bacteria to bind to and/or adhere to mucus at high affinity; bacterial surface antigens that retain their immunological characteristics thereby permitting efficient binding of primary antibodies used in ELISA and the killing and/or inactivation of the bacteria in such a way so as to not alter antibody binding; and/or bacteria preparation carried out in such a way so as to generate a formulation that has a long shelf life and allows simple and instant use in ELISA.

Modifications of the non-radioactive bacterial binding assay including the bacterial inoculum preparations of Example 18 were performed. Experiments were conducted using a less aggressive and more gentle (e.g., so as to preserve fimbriae and/or bacterial surface antigens (e.g., for antibody binding)) dual-kill procedure. As described below, a freeze dry procedure facilitated long term storage of the inoculum. For example, in some embodiments, the freeze dry procedure permitted the generation of single-use ampoules containing exactly the correct number of bacteria (e.g., for use with a mucus coated plate in an assay (e.g., for use in a kit)). These novel methods and compositions significantly reduced potential error on a technician's part (e.g., in having to determine the correct inoculum size), reduces risk of contamination of the inoculum and minimizes deterioration of the bacterial preparation over time. The freeze dried ampoules were stable at room temperature and are transportable (e.g., globally) without risk of loss of functionality.

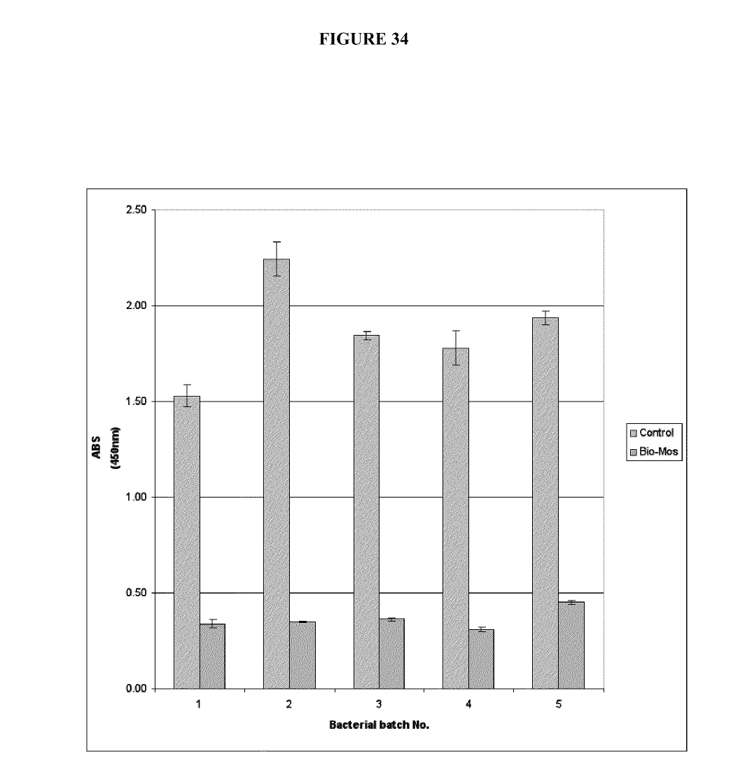

Bacterial preparation method. Bacteria (e.g., E. coli F4+ (former K88) strain) were grown at 37° C. in Luria Bertani broth. The cultures were harvested by centrifugation, re-suspended in saline solution and instantly enumerated by microscopic counting. Bacterial suspensions were killed by heating at 65° C. for 45 minutes followed by UV radiation for 45 minutes. Bacterial batches were then divided into ampoules, each containing 1×10⁹ bacterial cells and frozen at −80° C. After 24 hours of freezing the ampoules were freeze-dried and sealed. The ampoules were stored at +4° C. Viable E. coli in the ampoules was determined by two different approaches, direct plating and using Most Probable Number (MPN). Ten-fold serial dilutions were prepared in five replicates from each of the bacterial batches for direct plating. The medium used was a rich unselective Luria Berthani. Colonies were counted after 2 days incubation at 37° C. MPN was performed by serial dilution of the content of the ampoule directly in rich unselective nutrient broth. MPN was done in three replicates (3-table MPN). Growth in the MPN tubes was first recorded after 2 days at 37° C., and, then again after 2 weeks. In the MPN method the whole contents of the bacterial ampoule (˜10⁹ cells) were suspended in the first, least diluted tube. Thus, MPN provides that a single viable bacterium should be detected. In plate count, the contents of the ampoule were suspended in 10 ml of diluent, of which 0.1 ml was spread on plates. Thus, no growth represents that the ampoule contained less 100 viable E. coli cells. Five different batches of bacterial preparation (ampoules) were tested for viability. The results from both testing methods, direct plating and MPN enumeration, showed no signs of viability. Thus, the present invention provides a new dual-kill method that provides a highly reproducible and consistent bacterial preparation (e.g., as determined in the mucus adherence assay (See, e.g., FIG. 34)).

Production and Stabilization of mucus coated plates. Experiments were conducted during development of the invention in order to prepare a mucus coated plate that possessed the following characteristics: mucus coating of even quality between wells of each plate and between different plates; mucus plates that are stabilized in such a way that does not destroy the bacteria and/or binding characteristics of the mucus coated on the plates (e.g., that preserves mucus surface properties involved in binding bacteria); and/or mucus coated plates that are stable (e.g., at room temperature or colder) for long periods of time (e.g., days, weeks, months, a year or more) that can are ready to be used (e.g., in an ELISA).

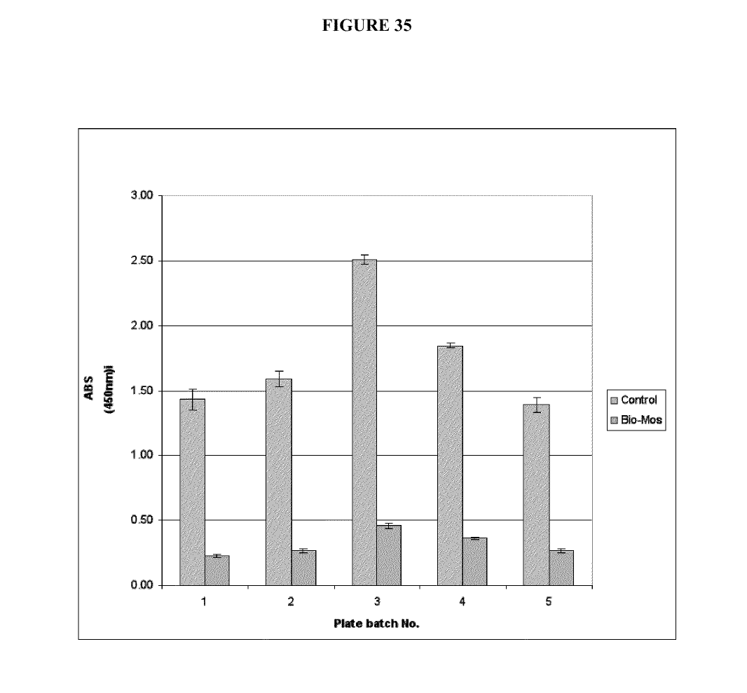

Mucus coated microtitre plates. Mucus was harvested from freshly slaughtered pigs by scraping the mucosa from the distal small intestine (ileum). Mucus was washed and clarified by centrifugation as described in Example 18. Mucus protein was quantified by using Bicinchoninic Acid Protein Assay kit from SIGMA (B9643). Nunc MaxiSorb plates (96-well format) were coated with mucus buffer solution containing 0.1 mg mucus protein/ml coating buffer. Batches of microtitre plates were independently prepared on different days and stored at +4° C. until testing day. The bacterial adherence assay, with and without Bio-Mos, was run in these plates to test plate-to-plate variation. Inhibition average between plates was 81.9%, from which the individual batches deviated in the average 1.5% (See, e.g., FIG. 35).

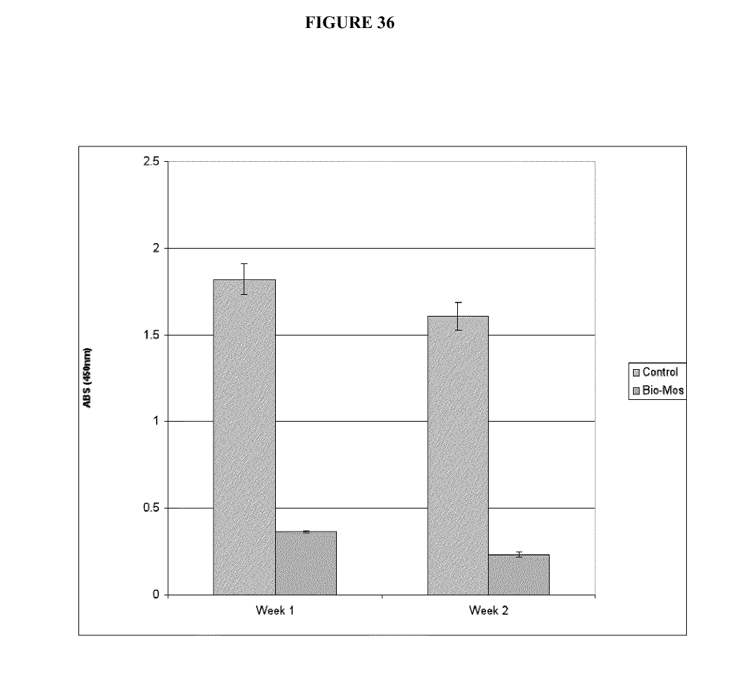

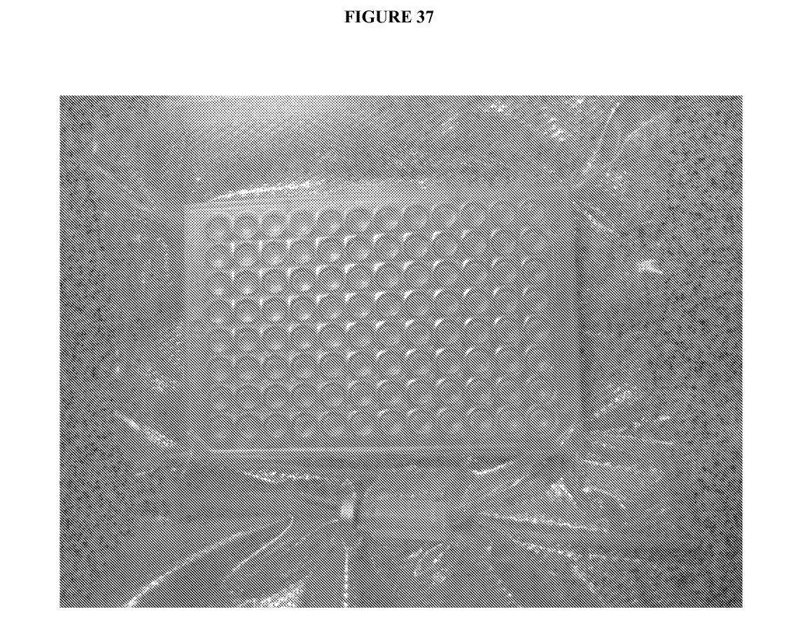

Additional experiments were performed during development of the invention to investigate the stability of the mucus plates when stored under mucus-buffer solution for 1 and 2 weeks in a vacuum seal package at 4° C. After two weeks of storage, the mucus plates were tested in order to determine if they could be used in an adherence assay with bacterial preparation. The results are shown in FIG. 36. The absolute signals in the assay show slight deterioration of intensity. However, in previous studies, variations were observed in intensity with respect to plate-to-plate variation and may have nothing to do with the storage of the plates. The application of the anti-adherence product, Bio-Mos, demonstrated that an approximate 80% inhibition was observed. Thus, the present invention provides methods of generating mucus coated plates, and the mucus coated plates themselves, that can be stored and utilized at a later time point (e.g., for adherence assays). For example, in some embodiments the present invention provides a mucus coated plate and/or a bacterial preparation (e.g., stored in an ampoule) that can either be stored individually or together (e.g., in a vacuum sealed package (See, e.g., FIG. 37)), that may be made commercially available for purchase and/or use (e.g., in an adherence assay).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A kit comprising a non-radioactive enzyme-linked immunosorbent assay (ELISA) for the assay of bacterial adherence and anti-adherence with mucus and/or epithelial cells comprising a solid support having mucus and/or epithelial cells coated thereon, a sample comprising bacteria, a primary antibody specific for said bacteria, and a detectably labeled secondary antibody specific for said primary antibody bound to said bacteria.

2. The kit of claim 1, further comprising a substrate which allows the visualization of the detectably labeled secondary antibody.

3. The kit of claim 2, wherein said detectably labeled secondary antibody comprises an enzyme label.

4. The kit of claim 3, wherein said substrate is a composition for providing a colorimetric, fluorimetric or chemiluminescent signal in the presence of said enzyme label.

5. The kit of claim 1, wherein said detectably labeled secondary antibody comprises pig anti-IgG immunoglobulins coupled to peroxidase.

6. The kit of claim 4, wherein said colorimetric composition is 3,3′,5,5′-tetramethylbenzidine.

7. The kit of claim 1, wherein said solid support is a 96-well plate.

8. The kit of claim 1, wherein said bacteria is E. coli bacteria.

9. The kit of claim 1, wherein said mucus is selected from the group consisting of pig proximal ileum mucus, pig distal colon mucus, broiler duodenum mucus and broiler caecum mucus.

10. The kit of claim 1, wherein said primary antibody is an horseradish peroxidase (HRP)-conjugated polyclonal antibody specific to E. coli O and K antigenic serotypes.

11. The kit of claim 1, wherein said primary antibody is a polyclonal antibody specific to E. coli O and K antigenic serotypes.

12. The kit of claim 1, wherein said secondary antibody is an affinity purified Rabbit anti-Goat IgG-HRP.

13. The kit of claim 1, wherein said secondary antibody is an affinity purified Rabbit anti-Goat IgG-AP.

14. The kit of claim 1, wherein said secondary antibody is a polyclonal FITC-conjugated antibody to Goat IgG.

15. The kit of claim 1, wherein secondary antibody is Streptavidin-Alkaline Phosphatase from Streptomyces avidinii.

16. The kit of claim 1, wherein secondary antibody is Streptavidin-Peroxidase from Streptomyces avidinii.

17. A method for measuring adherence and anti-adherence between bacteria and mucus and/or epithelial cells comprising
a) providing
i) a sample comprising bacteria; and
ii) mucus and/or epithelial, or other cells; and
b) combining said sample comprising bacteria and said mucus and/or epithelial cells within a non-radioactive colorimetric assay under conditions such that adherence and anti-adherence between said bacteria and said mucus and/or epithelial cells is measured.

18. The method of claim 17, wherein said non-radioactive colorimetric assay is an ELISA assay.

19. The method of claim 17, wherein said conditions comprise adding primary antibodies specific for said bacteria bound with mucus and/or epithelial cells.

20. The method of claim 19, wherein said conditions comprise adding detectably labeled secondary antibodies specific for said primary antibodies bound with said bacteria.

21. The method of claim 20, wherein said conditions comprise adding a substrate which allows the visualization of said detectably labeled secondary antibodies bound with said primary antibodies.

22. The method of claim 17, wherein said mucus and/or epithelial are coated onto a microtitre plate.

23. The method of claim 20, wherein said detectably labeled secondary antibody comprises an enzyme label.

24. The method of claim 21, wherein said substrate is a composition for providing a colorimetric, fluorimetric or chemiluminescent signal in the presence of said enzyme label.

25. The method of claim 24, wherein said detectably labeled secondary antibody comprises pig anti-IgG immunoglobulins coupled to peroxidase.

26. The method of claim 24, wherein said colorimetric composition is 3,3′,5,5′-tetramethylbenzidine.

27. The method of claim 17, wherein said bacteria is E. coli bacteria.

28. The method of claim 17, wherein said mucus is selected from the group consisting of pig proximal ileum mucus, pig distal colon mucus, broiler duodenum mucus and broiler caecum mucus.

29. The method of claim 19, wherein said primary antibody is an HRP-conjugated polyclonal antibody specific to E. coli O and K antigenic serotypes.

30. The method of claim 19, wherein said primary antibody is a polyclonal antibody specific to E. coli O and K antigenic serotypes.

31. The method of claim 20, wherein said secondary antibody is an affinity purified Rabbit anti-Goat IgG-HRP.

32. The method of claim 20, wherein said secondary antibody is an affinity purified Rabbit anti-Goat IgG-AP.

33. The method of claim 20, wherein said secondary antibody is a polyclonal FITC-conjugated antibody to Goat IgG.

34. The method of claim 20, wherein secondary antibody is Streptavidin-Alkaline Phosphatase from Streptomyces avidinii.

35. The method of claim 20, wherein secondary antibody is Streptavidin-Peroxidase from Streptomyces avidinii.

36. A method for identifying an agent that modulates adherence between bacteria and mucus and/or epithelial cells, comprising
a) providing
i) a sample comprising bacteria;
ii) mucus and/or epithelial cells; and
iii) an agent; and
b) combining said sample comprising bacteria, said mucus and/or epithelial cells, and said agent within a non-radioactive colorimetric assay under conditions such that adherence between said bacteria and said mucus and/or epithelial cells is measured;
c) comparing said bacterial adherence in the presence and absence of said agent; and
d) identifying said agent as a modulator of adherence between said bacteria and said mucus and/or epithelial cells if said measured adherence is higher or lower than adherence between said bacteria and said mucus and/or epithelialcells in the absence of said agent.

37. The method of claim 36, wherein said non-radioactive colorimetric assay is an ELISA assay.

38. The method of claim 36, wherein said conditions comprise adding primary antibodies specific for said bacteria bound with said mucus and/or epithelial cells.

39. The method of claim 38, wherein said conditions comprise adding detectably labeled secondary antibodies specific for said primary antibodies bound with said bacteria.

40. The method of claim 39, wherein said conditions comprise adding a substrate which allows the visualization of said detectably labeled secondary antibodies bound with said primary antibodies.

41. The method of claim 36, wherein said mucus is coated onto a microtitre plate.

42. The method of claim 39, wherein said detectably labeled secondary antibody comprises an enzyme label.

43. The method of claim 40, wherein said substrate is a composition for providing a colorimetric, fluorimetric or chemiluminescent signal in the presence of said enzyme label.

44. The method of claim 43, wherein said detectably labeled secondary antibody comprises pig anti-IgG immunoglobulins coupled to peroxidase.

45. The method of claim 43, wherein said colorimetric composition is 3,3′,5,5′-tetramethylbenzidine.

46. The method of claim 36, wherein said bacteria is E. coli bacteria.

47. The method of claim 36, wherein said mucus and/or epithelial cells are selected from the group consisting of pig proximal ileum mucus, pig distal colon mucus, broiler chicken duodenum mucus and broiler chicken caecum mucus.

48. The method of claim 38, wherein said primary antibody is an HRP-conjugated polyclonal antibody specific to E. coli O and K antigenic serotypes.

49. The method of claim 38, wherein said primary antibody is a polyclonal antibody specific to E. coli O and K antigenic serotypes.

50. The method of claim 39, wherein said secondary antibody is an affinity purified Rabbit anti-Goat IgG-HRP.

51. The method of claim 39, wherein said secondary antibody is an affinity purified Rabbit anti-Goat IgG-AP.

52. The method of claim 39, wherein said secondary antibody is a polyclonal FITC-conjugated antibody to Goat IgG.

53. The method of claim 39, wherein secondary antibody is Streptavidin-Alkaline Phosphatase from Streptomyces avidinii.

54. The method of claim 39, wherein secondary antibody is Streptavidin-Peroxidase from Streptomyces avidinii.

55. The method of claim 36, wherein said agent is selected from a list consisting of a naturally occuring molecule, a synthetically derived molecule, and a recombinantly derived molecule.

56. A composition comprising an agent, wherein said agent is a modulator of bacterial adherence with mucus and/or epithelial cells, wherein said agent is identified through a process comprising:
a) providing
i) a sample comprising bacteria;
ii) mucus and/or epithelial cells; and
iii) an agent; and
b) combining said sample comprising bacteria, said mucus and/or epithelial cells, and said agent within a non-radioactive colorimetric assay under conditions such that adherence between said bacteria and said mucus and/or epithelial cells is measured;
c) comparing said bacterial adherence in the presence and absence of said agent; and
d) identifying said agent as a modulator of adherence between said bacteria and said mucus and/or epithelial cells if said measured adherence is higher or lower than adherence between said bacteria and said mucus and/or epithelial cells in the absence of said agent.

57. The composition of claim 56, wherein said composition is within a foodstuff configured for consumption by a subject selected from the group consisting of livestock, animals, fish, and shellfish.
